(19) United States
(12) Patent Application Publication
(10) Pub. No.: US 2006/0269516 A1
(43) Pub. Date: Nov. 30, 2006

(54) Interferon-IgG fusion

(75) Inventors: Leonard G. Presta, San Francisco, CA (US); Narendra Kishnani, East Brunswick, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/439,555

(22) Filed: May 24, 2006

Related U.S. Application Data
(60) Provisional application No. 60/685,018, filed on May 26, 2005.

(51) Int. Cl. A61K 38/21; C07K 14/56; C07K 14/57; C07K 16/46

(52) U.S. Cl. 424/85.4; 424/85.5; 424/85.6; 424/85.7; 530/351; 530/391.1

(57) ABSTRACT

The present invention provides, inter alia, polypeptides for the treatment of various diseases such as HCV as well as methods of treatment and methods of making the polypeptides.

The present application claims the benefit of U.S. provisional patent application No. 60/685,018; filed May 26, 2005, which is herein incorporated by reference in its entirety.

### FIELD OF THE INVENTION

The present invention relates to fusion polypeptides between interferon (IFN) and IgG4 as well as methods of use and methods of production thereof.

### BACKGROUND OF THE INVENTION

Various types of interferon have been approved for treatment of viral infections, cancers and other diseases including multiple sclerosis. For example, interferon alfa-2b has been approved for hairy cell leukemia, malignant melanoma, follicular lymphoma, condylomata acuminate, AIDS-related kaposi's sarcoma, chronic hepatitis C infection and chronic hepatitis B infection. An advantage of fusing interferon to polyethylene glycol (PEG) is to increase its in vivo half-life and, thereby, reduce the number of doses required over time. For example, two such IFN pegylated products have been approved with a dosing frequency of once a week: PEG-Intron® and Pegasys® which respectively have a 12 kDa PEG or 40 kDa PEG covalently linked to the IFN protein. Besides improving patient compliance, the pharmacokinetic extension in the half-life favorably alters the pharmocodynamic characteristics and related efficacies of therapy as prolonged sustained systemic circulating levels of IFN result in better efficacy than pulsatile profiles. Jones et al. (J. Interferon and Cytokine Res. 24:560-572 (2004)), describe a fusion between interferon-alfa-2b and mutants thereof and IgG1. Jones et al. claim that the fusion may be suitable for treatment of HCV infection and that it possesses beneficial pharmacokinetic properties. Because, for example, IgG1 has been demonstrated to exhibit a relatively high level of antibody dependent cell-mediated cytotoxicity (ADCC) (see Steplewski et al., Proc Natl Acad Sci USA. 85(13):4852-4856 (1988)), there exists a need in the art for a composition comprising interferon fused to a molecule that exhibits an extended half-life, low toxicity and high activity and is simple and cheap to manufacture.

### SUMMARY OF THE INVENTION

The present invention addresses, inter alia, this need in the art. The present invention provides an isolated polypeptide comprising one or more interferon polypeptides fused to one or more IgG4 polypeptides. In an embodiment, the interferon is a member selected from the group consisting of interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon gamma, interferon alpha F and interferon alpha con1 (e.g., SEQ ID NO: 2, 3 or 15). In an embodiment, the interferon comprises an amino acid sequence selected from the group consisting of SEQ ID NOs: 12, 13 and 14. In an embodiment, the IgG4 comprises an amino acid sequence set forth in SEQ ID NO: 1. In an embodiment, the interferon is fused to the IgG4 by a peptide linker (e.g., wherein the linker comprises from about 2 to about 18 amino acids (e.g., 2, 3, 4, 5, 6, 7, 8, 9, 10, 11, 12, 13, 14, 15, 16, 17, 18); e.g., comprising the amino acid sequence of SEQ ID NO: 7, 8, 9, 10, 11, 17, 18, 19 or 20). The present invention also comprises any of the IFN-IgG4 fusions herein in a pharmaceutical composition comprising a pharmaceutically acceptable carrier.

The present invention also provides a multimer comprising two or more (e.g., 2, 3, 4, 5, 6, 7, 8, 9 or 10) IFN-Ig polypeptides of the invention bound together in a non-covalent complex. In an embodiment of the invention, the polypeptides of the multimer are coordinated with a divalent cation such as Zn²⁺.

The present invention also provides any of the IFN-Ig polypeptide herein in crystalline form.

Included within the scope of the present invention is a composition comprising any IFN-IgG4 fusion herein in association with one or more further pharmaceutical agents; for example, suitable for treating a medical condition selected from the group consisting of Flaviviridae virus infection, multiple sclerosis, serious infections associated with chronic granulomatous disease, malignant osteopetrosis, refractory or recurring external condylomata acuminate, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, hepatitis B infection and hepatitis C infection. In an embodiment, the further agent is a member selected from the group consisting of ribavirin, isatoribine, VX-497, viramidine, BILN 2061, VX-950, IDN-6556 and any other agent set forth herein, for example, under the “Pharmaceutical Compositions” section below or a pharmaceutical composition thereof.

The present invention also includes an isolated polynucleotide encoding any IFN-IgG4 fusion set forth herein. In an embodiment of the invention, the polynucleotide comprises a nucleotide sequence selected from the group consisting of SEQ ID NOs: 4, 5 and 16. An embodiment of the invention includes a recombinant vector comprising a polynucleotide of the invention. Also included within the scope of the present invention is an isolated host cell comprising the vector.

The present method provides a method for increasing the in vivo half-life of interferon comprising fusing the interferon to IgG4 (e.g., to create a fusion comprising an amino acid sequence selected from SEQ ID NOs: 2, 3 and 15). For example, in an embodiment, the method comprises recombinantly expressing the fusion polypeptide, for example, in a host cell (e.g., a bacterial cell such as E. coli); for example, wherein the fusion is expressed by introducing a polynucleotide encoding the fusion, for example, in a recombinant vector operably associated with a regulatory element such as a promoter, under conditions wherein the fusion is expressed, optionally isolating the fusion, and introducing the fusion or a pharmaceutical composition thereof into the body of a subject, such as a human. In an embodiment, the interferon is a member selected from the group consisting of interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon gamma, interferon alpha F and interferon alpha con 1. In an embodiment, the interferon comprises an amino acid sequence selected from the group consisting of SEQ ID NOs: 12, 13 and 14. In an embodiment, the IgG4 comprises an amino acid sequence set forth in SEQ ID NO: 1.

The present invention also provides a method for treating or preventing, in a subject, any medical condition that is treatable by interferon therapy, for example, selected from the group consisting of Flaviviridae virus infection, multiple sclerosis, serious infections associated with chronic granulomatous disease, malignant osteopetrosis, refractory or recurring external condylomata acuminate, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, hepatitis B infection and hepatitis C infection, comprising administering, to the subject, a therapeutically effective amount of an isolated polypeptide comprising interferon fused to IgG4 or a pharmaceutically acceptable composition thereof (e.g., comprising an amino acid sequence selected from SEQ ID NOs: 2, 3 and 15). In an embodiment, the subject is pregnant or a nursing mother. In an embodiment, the interferon is a member selected from the group consisting of is interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon gamma, interferon alpha F and interferon alpha con1. In an embodiment, the polypeptide is administered in association with one or more further pharmaceutical agents; for example, suitable for treating a medical condition selected from the group consisting of Flaviviridae virus infection, multiple sclerosis, serious infections associated with chronic granulomatous disease, malignant osteopetrosis, refractory or recurring external condylomata acuminate, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, hepatitis B infection and hepatitis C infection or a pharmaceutical composition thereof. In an embodiment, the additional agent is selected from the group consisting of ribavirin, isatoribine, VX-497, viramidine, BILN 2061, VX-950, IDN-6556 and any other agent set forth herein, for example, under the “Pharmaceutical Compositions” section below. In an embodiment, the interferon comprises an amino acid sequence selected from the group consisting of SEQ ID NOs: 12, 13 and 14. In an embodiment, the IgG4 comprises an amino acid sequence set forth in SEQ ID NO: 1. In an embodiment, the host is a human (e.g., a pregnant human or nursing mother). In an embodiment, the therapeutically effective amount of the isolated polypeptide comprising interferon fused to IgG4, optionally in association with an anti-viral therapeutic, or the pharmaceutically acceptable composition thereof is administered for a treatment time period sufficient to eradicate detectable hepatitic C virus-RNA and to maintain no detectable hepatitic C virus RNA for at least twelve weeks (e.g., 24 weeks) after the end of the treatment time period

The present invention also provides a method for making a polypeptide comprising interferon fused to IgG4 comprising introducing a polynucleotide encoding said polypeptide into a host cell under conditions wherein the polynucleotide is expressed. In an embodiment, the method further comprises isolating the polypeptide. Also within the scope of the present invention is any polypeptide produced by the foregoing method for making a polypeptide.

A further embodiment of the invention includes any isolated fusion comprising IgG4 fused, optionally by a linker peptide, to a short half-life cytokine (e.g., IL-10 from any species); along with any polynucleotide encoding such a fusion, any insolated vector comprising such a polynucleotide and any host cell comprising such a vector. The present invention also comprises any method for treating or preventing any inflammatory disorder (e.g., multiple sclerosis, inflammatory bowel syndrome, psoriasis, Crohn's disease, rheumatoid arthritis, or ulcerative colitis) in a subject by administering a therapeutically effective amount of an IgG4-IL-10 fusion polypeptide.

### DETAILED DESCRIPTION OF THE INVENTION

The present invention provides, inter alia, an IFN-IgG4 product that exhibits a beneficial in vivo PK/PD profile and that can be conveniently made with a single-step manufacturing process. Indeed, the single-step manufacturing process is of similar complexity to that of conventional recombinant expression of IFN. Moreover, the PK/PD profile of IFN-IgG4 requires only a low dosing frequency. IgG4 is beneficial, as compared to other IgGs, such as IgG1, because it exhibits low toxicity such as ADCC (antibody-dependent cell-mediated cytotoxicity) and/or CDC (complement-dependent cytotoxicity). Without being bound by a single theory or mechanism of action, the IgG4 fusions of the invention exhibit lower ADCC and/or CDC than other immunoglobulin subtypes because IgG4 binds Fc complement and/or Fc gamma receptors relatively poorly (see e.g., Steplewski et al., Proc Natl Acad Sci USA. 85(13):4852-4856 (1988)). Without being bound by a single theory or mechanism of action, the fusions of the present invention would be suitable for treatment of pregnant women because the fusion does not cross the placental barrier efficiently. Interferon a2b has been shown to have abortive effects in Macaca mulatta (rhesus monkeys) at 15 and 30 million IU/kg. An interferon a2b which cannot cross the placental barrier and is not contraindicated for pregnant woman with e.g., a viral infection (e.g., hepatitis C virus) would be beneficial.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A “polynucleotide”, “nucleic acid” or “nucleic acid molecule” includes the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in single stranded form, double-stranded form or otherwise.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

A “coding sequence” or a sequence “encoding” an expression product, such as a RNA, polypeptide, protein, or enzyme, is a nucleotide sequence that, when expressed, results in production of the product.

The term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

A “protein sequence”, “peptide sequence” or “polypeptide sequence” or “amino acid sequence” includes a series of two or more amino acids in a protein, peptide or polypeptide.

The terms “isolated polynucleotide” or “isolated polypeptide” include a polynucleotide (e.g., RNA or DNA molecule, or a mixed polymer) or a polypeptide, respectively, which are partially or fully separated from other components that are normally found in cells or in recombinant DNA expression systems. These components include, but are not limited to, cell membranes, cell walls, ribosomes, polymerases, serum components and extraneous genomic sequences.

An isolated polynucleotide or polypeptide will, preferably, be an essentially homogeneous composition of molecules but may contain some heterogeneity.

“Amplification” of DNA as used herein includes the use of polymerase chain reaction (PCR) to increase the concentration of a particular DNA sequence within a mixture of DNA sequences. For a description of PCR see Saiki, et al., Science (1988) 239:487.

The term “host cell” includes any cell of any organism that is selected, modified, transfected, transformed, grown, or used or manipulated in any way, for the production of a substance by the cell, for example the expression or replication, by the cell, of a gene, a DNA or RNA sequence or a protein. Host cells include bacterial cells (e.g., E. coli), Chinese hamster ovary (CHO) cells, murine macrophage J774 cells or any other macrophage cell line and human intestinal epithelial Caco2 cells.

The nucleotide sequence of a polynucleotide may be determined by any method known in the art (e.g., chemical sequencing or enzymatic sequencing). “Chemical sequencing” of DNA includes methods such as that of Maxam and Gilbert (1977) (Proc. Natl. Acad. Sci. USA 74:560), in which DNA is randomly cleaved using individual base-specific reactions. “Enzymatic sequencing” of DNA includes methods such as that of Sanger (Sanger, et al., (1977) Proc. Natl. Acad. Sci. USA 74:5463).

The polynucleotides herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

In general, a “promoter” or “promoter sequence” is a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a polynucleotide of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the β-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

A coding sequence is “under the control of”, “functionally associated with” or “operably associated with” transcriptional and translational control sequences in a cell when the sequences direct RNA polymerase mediated transcription of the coding sequence into RNA, preferably mRNA, which then may be RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “transformation” means the introduction of polynucleotide into a cell. The introduced gene or sequence may be called a “clone”. A host cell that receives the introduced DNA or RNA has been “transformed” and is a “transformant” or a “clone.” The DNA or RNA introduced to a host cell can come from any source, including cells of the same genus or species as the host cell, or from cells of a different genus or species.

The term “vector” includes a vehicle (e.g., a plasmid) by which a DNA or RNA sequence can be introduced into a host cell, so as to transform the host and, optionally, promote expression and/or replication of the introduced sequence.

Vectors that can be used in this invention include plasmids, viruses, bacteriophage, integratable DNA fragments, and other vehicles that may facilitate introduction of the polynucleotide into the host. Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

The term “expression system” means a host cell and compatible vector which, under suitable conditions, can express a protein or nucleic acid which is carried by the vector and introduced to the host cell. Common expression systems include E. coli host cells and plasmid vectors, insect host cells and Baculovirus vectors, and mammalian host cells and vectors.

Expression of nucleic acids encoding an IFN-IgG4 fusion polypeptide of this invention can be carried out by conventional methods in either prokaryotic or eukaryotic cells. Although E. coli host cells are employed most frequently in prokaryotic systems, many other bacteria, such as various strains of Pseudomonas and Bacillus, are known in the art and can be used as well. Suitable host cells for expressing nucleic acids encoding the IFN-IgG4 fusion polypeptides include prokaryotes and higher eukaryotes. Prokaryotes include both gram-negative and gram-positive organisms, e.g., E. coli and B. subtilis. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. A representative vector for amplifying DNA is pBR322 or any of many of its derivatives (e.g., pUC18 or 19). Vectors that can be used to express the IFN-IgG4 polypeptides include, but are not limited to, those containing the lac promoter (pUC-series); trp promoter (pBR322-trp); lpp promoter (the pIN-series); lambda-pP or pR promoters (pOTS); or hybrid promoters such as ptac (pDR540). See Brosius et al., “Expression Vectors Employing Lambda-, trp-, lac-, and lpp-derived Promoters”, in Rodriguez and Denhardt (eds.) Vectors: A Survey of Molecular Cloning Vectors and Their Uses, 1988, Buttersworth, Boston, pp. 205-236. Many polypeptides can be expressed, at high levels, in an E. coli/T7 expression system as disclosed in U.S. Pat. Nos. 4,952,496, 5,693,489 and 5,869,320 and in Davanloo, P., et al., (1984) Proc. Natl. Acad. Sci. USA 81: 2035-2039; Studier, F. W., et al., (1986) J. Mol. Biol. 189: 113-130; Rosenberg, A. H., et al., (1987) Gene 56: 125-135; and Dunn, J. J., et al., (1988) Gene 68: 259.

Higher eukaryotic tissue culture cells may also be used for the recombinant production of the IFN-IgG4 fusion polypeptides of the invention. Higher eukaryotic tissue culture cell line can be used, including insect baculovirus expression systems and mammalian cells. Transformation or transfection and propagation of such cells have become a routine procedure. Examples of useful cell lines include HeLa cells, Chinese hamster ovary (CHO) cell lines, J774 cells, Caco2 cells, baby rat kidney (BRK) cell lines, insect cell lines, bird cell lines, and monkey (COS) cell lines. Typically, expression vectors for such cell lines include an origin of replication, a promoter, a translation initiation site, RNA splice sites (if genomic DNA is used), a polyadenylation site, and a transcription termination site. These vectors also, usually, contain a selection gene or amplification gene. Suitable expression vectors may be plasmids, viruses, or retroviruses carrying promoters derived, e.g., from such sources as adenovirus, SV40, parvoviruses, vaccinia virus, or cytomegalovirus. Examples of expression vectors include pCR®3.1, pCDNA1, pCD (Okayama, et al., (1985) Mol. Cell Biol. 5:1136), pMC1neo Poly-A (Thomas, et al., (1987) Cell 51:503), pREP8, pSVSPORT and derivatives thereof, and baculovirus vectors such as pAC373 or pAC610.

Modifications (e.g., post-translational modifications) that occur in a polypeptide often will be a function of how it is made. For polypeptides made by expressing a cloned gene in a host, for instance, the nature and extent of the modifications, in large part, will be determined by the host cell's post-translational modification capacity and the modification signals present in the polypeptide amino acid sequence. For instance, as is well known, glycosylation often does not occur in bacterial hosts such as E. coli. Accordingly, when glycosylation of IFN-IgG4 is desired, the polypeptide can be expressed in a glycosylating host, generally a eukaryotic cell. Insect cells often carry out post-translational glycosylations which are similar to those of mammalian cells. For this reason, insect cell expression systems have been developed to express, efficiently, mammalian proteins having native patterns of glycosylation. An insect cell which may be used in this invention is any cell derived from an organism of the class Insecta; for example, where the insect is Spodoptera fruigiperda (Sf9 or Sf21) or Trichoplusia ni (High 5). Examples of insect expression systems that can be used with the present invention, for example to produce an IFN-IgG4 fusion polypeptide, include Bac-To-Bac (Invitrogen Corporation, Carlsbad, Calif.) or Gateway (Invitrogen Corporation, Carlsbad, Calif.). If desired, deglycosylation enzymes can be used to remove carbohydrates attached during production in eukaryotic expression systems.

The present invention contemplates any superficial or slight modification to the amino acid or nucleotide sequences which correspond to the IFN-IgG4 polypeptides of the invention. In particular, the present invention contemplates sequence conservative variants of the polynucleotides which encode the polypeptides of the invention. “Sequence-conservative variants” of a polynucleotide sequence are those in which a change of one or more nucleotides in a given codon results in no alteration in the amino acid encoded at that position. Function-conservative variants of the polypeptides of the invention are also contemplated by the present invention. “Function-conservative variants” are those in which one or more amino acid residues in a protein or enzyme have been changed without altering the overall conformation and function of the polypeptide, including, but, by no means, limited to, replacement of an amino acid with one having similar properties. Amino acids with similar properties are well known in the art. For example, polar/hydrophilic amino acids which may be interchangeable include asparagine, glutamine, serine, cysteine, threonine, lysine, arginine, histidine, aspartic acid and glutamic acid; nonpolar/hydrophobic amino acids which may be interchangeable include glycine, alanine, valine, leucine, isoleucine, proline, tyrosine, phenylalanine, tryptophan and methionine; acidic amino acids which may be interchangeable include aspartic acid and glutamic acid and basic amino acids which may be interchangeable include histidine, lysine and arginine.

The present invention includes polynucleotides encoding an IFN-IgG4 fusion polypeptide (e.g., SEQ ID NO: 4, 5 or 16) as well as nucleic acids which hybridize to the polynucleotides. Preferably, the nucleic acids hybridize under low stringency conditions, more preferably under moderate stringency conditions and most preferably under high stringency conditions. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook, et al., supra). The conditions of temperature and ionic strength determine the “stringency” of the hybridization. Typical low stringency hybridization conditions are 55° C., 5×SSC, 0.1% SDS, 0.25% milk, and no formamide at 42° C.; or 30% formamide, 5×SSC, 0.5% SDS at 42° C. Typical, moderate stringency hybridization conditions are similar to the low stringency conditions except the hybridization is carried out in 40% formamide, with 5× or 6×SSC at 42° C. High stringency hybridization conditions are similar to low stringency conditions except the hybridization conditions are carried out in 50% formamide, 5× or 6×SSC and, optionally, at a higher temperature (e.g., higher than 42° C.: 57° C., 59° C., 60° C., 62° C., 63° C., 65° C. or 68° C.). In general, SSC is 0.15M NaC1 and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, i.e., oligonucleotides, the position of mismatches becomes more important, and the length of the oligonucleotide determines its specificity (see Sambrook, et al., supra).

Also included in the present invention are polynucleotides comprising nucleotide sequences and polypeptides comprising amino acid sequences which are at least about 70% identical, preferably at least about 80% identical, more preferably at least about 90% identical and most preferably at least about 95% identical (e.g., 95%, 96%, 97%, 98%, 99%, 100%) to the reference IFN-IgG4 fusion polynucleotide of any of SEQ ID NOs: 4, 5, or 16 and amino acid sequence of any of SEQ ID NOs: 2, 3, or 15 when the comparison is performed by a BLAST algorithm wherein the parameters of the algorithm are selected to give the largest match between the respective sequences over the entire length of the respective reference sequences. Polypeptides comprising amino acid sequences which are at least about 70% similar, preferably at least about 80% similar, more preferably at least about 90% similar and most preferably at least about 95% similar (e.g., 95%, 96%, 97%, 98%, 99%, 100%) to the reference IFN-IgG4 fusion amino acid sequence of any of SEQ ID NOs: 2, 3 or 15, when the comparison is performed with a BLAST algorithm wherein the parameters of the algorithm are selected to give the largest match between the respective sequences over the entire length of the respective reference sequences, are also included in the present invention.

Sequence identity refers to exact matches between the nucleotides or amino acids of two sequences which are being compared. Sequence similarity refers to both exact matches between the amino acids of two polypeptides which are being compared in addition to matches between nonidentical, biochemically related amino acids. Biochemically related amino acids which share similar properties and may be interchangeable are discussed above.

The following references regarding the BLAST algorithm are herein incorporated by reference: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M., et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### Fusions

The present invention comprises any fusion polypeptide comprising one or more of any interferon polypeptides, fused, optionally by a linker peptide, to one or more IgG4 Fc polypeptides (“IFN-IgG4 fusion”). In an embodiment, the interferon is interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon alpha-F or interferon alpha con1 or any interferon species disclosed in Blatt et al., J. Interferon and Cytokine Res. 16: 489-499 (1996) or in Pestka, Interferon from 1981 to 1986, Methods Enzymol. 119:3-14 (1986); which are herein incorporated by reference in their entireties. The polypeptide and encoding polynucleotide sequence for each of these interferon species is known in the art (see e.g., Allen et al., J. Interferon and Cytokine Res. 16:181-184 (1996); or Published U.S. Patent Application No. U.S. 2004/0219131A1; each of which is herein incorporated by reference).

In an embodiment of the invention, the amino acid sequence of human interferon alfa-2b is:

MCDLPQTHSLGSRRTLMLLAQMRRISLFSCLKDRHDFGFPQEEFGNQFQKAETIPVLHEMIQQIFNLFS

TKDSSAAWDETLLDKFYTELYQQLNDLEACVIQGVGVTETPLMKEDSILAVRKYFQRITLYLKEKKYSP

CAWEVVRAEIMRSFSLSTNLQESLRSKE

(SEQ ID NO: 12; see also Genbank accession no. CAA25770 ); wherein the first M is optional.

In an embodiment of the invention, and the amino acid sequence of human interferon alfa-2a is:

MCDLPQTHSLGSRRTLMLLAQMRKISLFSCLKDRHDFGFPQEEFGNQFQKAETIPVLHEMIQQIFNLFS

TKDSSAAWDETLLDKFYTELYQQLNDLEACVIQGVGVTETPLMKEDSILAVRKYFQRITLYLKEKKYSP

CAWEVVRAEIMRSFSLSTNLQESLRSKE

(SEQ ID NO: 13; see also Genbank accession no. 1ITF); wherein the first M is optional.

In an embodiment of the invention, the amino acid sequence of human consensus interferon alpha (alpha con-1) is:

MCDLPQTHSLGNRRALILLAQMRRISPFSCLKDRHDFGFPQEEFDGNQFQKAQAISVLHEMIQQTFNLF

STKDSSAAWDESLLEKFYTELYQQLNDLEACVIQEVGVEETPLMNVDSILAVKKYFQRITLYLTEKK

YSPCAWEVVRAEIMRSFSLSTNLQERLRRKE

(SEQ ID NO: 14; see Blatt et al., J. Interferon Cytokine Res. 16: 489-499 (1996); Klein et al., J. Chromatography 454: 205-215 (1988)); wherein the first M is optional.

The fusions of the invention comprise one or more interferons and/or one or more IgG4s. If the fusion comprises multiple interferons, the interferons may be the same or different. For example, a fusion of the invention comprises, in an embodiment, human interferon alfa-2a-human interferon alfa-2a-IgG4. In another embodiment, the fusion comprises human interferon alfa-2a-human interferon alfa-2b-IgG4 or interferon alfa-2a-IgG4-interferon alfa-2a. Multiple IgG4 polypeptides may also be included in a fusion of the invention. For example, in an embodiment, the fusion comprises human interferon alfa-2a-human interferon alfa-2a-IgG4-IgG4 or human interferon alfa-2a-human interferon alfa-2b-IgG4-IgG4. Any of these embodiments are included under the term “IFN-IgG4”.

Fusions comprising interferon at the amino-terminus are within the scope of the present invention along with fusions with interferon at the carboxy-terminus; the term IFN-IgG4 refers to both of these types of fusions. For example, the present invention comprises any of the following IFN-IgG4 fusions: IgG4 Fc-interferon alfa-1a, IgG4 Fc-interferon alfa-1b, IgG4 Fc-interferon alfa-2a, IgG4 Fc-interferon alfa-2b, IgG4 Fc-interferon alfa-2c, IgG4 Fc-interferon alfa-4a, IgG4 Fc-interferon alfa-4b, IgG4 Fc-interferon alfa-5, IgG4 Fc-interferon alfa-6, IgG4 Fc-interferon alfa-7a, IgG4 Fc-interferon alfa-7b, IgG4 Fc-interferon alfa-8a, IgG4 Fc-interferon alfa-8b, IgG4 Fc-interferon alfa-8c, IgG4 Fc-interferon alfa-10a, IgG4 Fc-interferon alfa-10b, IgG4 Fc-interferon alfa-13, IgG4 Fc-interferon alfa-14a, IgG4 Fc-interferon alfa-14b, IgG4 Fc-interferon alfa-14c, IgG4 Fc-interferon alfa-16, IgG4 Fc-interferon alfa-17a, IgG4 Fc-interferon alfa-17b, IgG4 Fc-interferon alfa-17c, IgG4 Fc-interferon alfa-17d, IgG4 Fc-interferon alfa-21a, IgG4 Fc-interferon alfa-21b, IgG4 Fc-interferon alfa-24, IgG4 Fc-interferon beta, IgG4 Fc-interferon omega, IgG4 Fc-interferon tau, IgG4 Fc-interferon alfa-N3, IgG4 Fc-interferon alfa-N, IgG4 Fc-interferon beta-1a, IgG4 Fc-interferon beta-1b, IgG4 Fc-interferon gamma-1b, IgG4 Fc-interferon gamma, IgG4 Fc-interferon alpha F, IgG4 Fc-interferon alpha con1, interferon alfa-1a-IgG4 Fc, interferon alfa-1b-IgG4 Fc, interferon alfa-2a-IgG4 Fc, interferon alfa-2b-IgG4 Fc, interferon alfa-2c-IgG4 Fc, interferon alfa-4a-IgG4 Fc, interferon alfa-4b-IgG4 Fc, interferon alfa-5-IgG4 Fc, interferon alfa-6-IgG4 Fc, interferon alfa-7a-IgG4 Fc, interferon alfa-7b-IgG4 Fc, interferon alfa-8a-IgG4 Fc, interferon alfa-8b-IgG4 Fc, interferon alfa-8c-IgG4 Fc, interferon alfa-10a-IgG4 Fc, interferon alfa-10b-IgG4 Fc, interferon alfa-13-IgG4 Fc, interferon alfa-14a-IgG4 Fc, interferon alfa-14b-IgG4 Fc, interferon alfa-14c-IgG4 Fc, interferon alfa-16-IgG4 Fc, interferon alfa-17a-IgG4 Fc, interferon alfa-17b-IgG4 Fc, interferon alfa-17c-IgG4 Fc, interferon alfa-17d-IgG4 Fc, interferon alfa-21a-IgG4 Fc, interferon alfa-21b-IgG4 Fc, interferon alfa-24-IgG4 Fc, interferon beta-IgG4 Fc, interferon omega-IgG4 Fc, interferon tau-IgG4 Fc, interferon alfa-N3-IgG4 Fc, interferon alfa-N-IgG4 Fc, interferon beta-1a-IgG4 Fc, interferon beta-1b-IgG4 Fc, interferon gamma-1b-IgG4 Fc, interferon gamma-IgG4 Fc, interferon alpha F-IgG4 Fc or interferon alpha con1-IgG4 Fc; wherein the interferon moiety and the IgG4 Fc moiety is optionally fused by a peptide linker; along with polynucleotides encoding any of the fusions of the invention.

In an embodiment, the IgG4 Fc comprises the following amino acid sequence

(SEQ ID NO: 1)

DKTHTCPPCPAPEFLGGPSVFLFPPKPKDTLMISRTPEVTCVVVDVSQED
PEVQFNWYVDGVEVHNAKTKPREEQFISISTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
optionally comprising a first methionine (M).

In an embodiment, the human interferon-alfa-2b-human IgG4 Fc fusion protein comprises the following amino acid sequence:

(SEQ ID NO: 2)

MALTFALLVALLVLSCKSSCSVGCDLPQTHSLGSRRTLMLLAQMRRISLF
SCLKDRHDFGFPQEEFGNQFQDAETIPVLHEMIQQIFNLFSTKDSSAAWD
ETLLDKFYTELYQQLNDLEACVIQGVGVTETPLMKEDSILAVRKYFQRIT
LYLKEKKYSPCAWEVVRAEIMRSFSLSTNLQESLRSKEASDKTHTCPPCP
APEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSKIAVE
WESNGQPENNYKTTPPVLDSDGSFFLYSRLTVDKSRWQEGNVFSCSVMHE
ALHNHYTQKSLSLSLGK;

wherein the signal sequence (MALTFALLVALLVLSCKSSCSVG) (SEQ ID NO: 6) is optional and so may be absent; however, in an embodiment of the invention, the N-terminus of the fusion optionally comprises a methionine (M); wherein the linker is underscored and is also optional; wherein the sequence that is N-terminal to the linker is human IFN-alfa-2b and the sequence that is C-terminal to the linker is human IgG4 Fc. In an embodiment, the linker is selected from SEQ ID NOs: 7-11 and 17-20. For example, in an embodiment, the interferon alfa-2b amino acid sequence is identical to that of Intron A® (Schering Corp.; Kenilworth, N.J.).

In an embodiment, the human interferon-alfa-2a-human IgG4 Fc fusion protein comprises the following amino acid sequence:

(SEQ ID NO: 3)

MCDLPQTHSLGSRRTLMLLAQMRKISLFSCLKDRHDFGFPQEEFGNQFQK
AETIPVLHEMIQQIFNLFSTKDSSAAWDETLLDKFYTELYQQLNDLEACV
IQGVGVTETPLMKEDSILAVRKYFQRITLYLKEKKYSPCAWEVVRAEIMR
SFSLSTNLQESLRSKEASDKTHTCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKBYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK;

wherein, in an embodiment of the invention, the N-terminus of the fusion optionally comprises a methionine (M); wherein the linker is underscored and is also optional; optionally comprising a signal sequence; wherein the sequence that is N-terminal to the linker is human IFN-alfa-2a and the sequence that is C-terminal to the linker is human IgG4 Fc. In an embodiment, the linker is selected from SEQ ID NOs: 7-11 and 17-20. For example, in an embodiment, the interferon alfa-2a amino acid sequence is identical to that of Roferon A® (Roche Laboratories; Nutley, N.J.).

In an embodiment, the human interferon-alpha con-1-human IgG4 Fc fusion protein comprises the following amino acid sequence:

(SEQ ID NO: 15)

MCDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEAC
VIQEVGVEETPLMNVDSILAVKKYFQRITLYLTEKKYSPCAWEVVRAEIM
RSFSLSTNLQERLRRKEASDKTHTCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;

wherein, in an embodiment of the invention, the N-terminus of the fusion optionally comprises a methionine (M); wherein the linker is underscored and is also optional; optionally comprising a signal sequence; wherein the sequence that is N-terminal to the linker is human IFN-alpha con-1 and the sequence that is C-terminal to the linker is human IgG4 Fc. In an embodiment, the linker is selected from SEQ ID NOs: 7-11 and 17-20.

The present invention includes any polynucleotide encoding any of the IFN-IgG4 fusions herein. In an embodiment, the polynucleotide encodes human interferon-alfa-2b-human IgG4 Fc comprising the following nucleotide sequence:

(SEQ ID NO: 4)

ATGGCNYTNA CNTTYGCNYT NYTNGTNGCN YTNYTNGTNY
TNWSNTGYAA RWSNWSNTGY WSNGTNGGNT GYGAYYTNCC
NCARACNCAY WSNYTNGGNW SNNGNMGNAC NYTNATGYTN
YTNGCNCARA TGMGNMGNAT HWSNYTNTTY WSNTGYYTNA
ARGAYMGNCA YGAYTTYGGN TTYCCNCARG ARGARTTYGG
NAAYCARTTY CARAARGCNG ARACNATHCC NGTNYTNCAY
GARATGATHC ARCARATHTT YAAYYTNTTY WSNACNAARG
AYWSNWSNGC NGCNTGGGAY GAPACNYTNY ThGAYAARTT
YTAYACNGAR YTNTAYCARC ARYTNAAYGA YYTNGARGCN
TGYGTNATHC ARGGNGTNGG NGTNACNGAR ACNCCNYTNA
TGAARGARGA YWSNATHYTN GCNGTNMGNA ARTAYTTYCA
RMGNATHACN YTNTAYYTNA ARGARAARAA RTAYWSNCCN
TGYGCNTGGG ARGTNGTNMG NGCNGARATH ATGMGNWSNT
TYWSNYTNWS NACNAAYYTN CARGARWSNY TNNGNWSNAA
RGARGCNWSN GAYAARACNC AYACNTGYCC NCCNTGYCCN
GCNCCNGART TYYTNGGNGG NCCNWSNGTN TTYYTNTTYC
CNCCNAARCC NAARGAYACN YTNATGATMW SNMGNACNCC
NGARGTNACN TGYGTNGTNG TNGAYGTNWS NCARGARGAY
CCNGARGTNC ARTTYAAYTG GTAYGTNGAY GGNGTNGARG
TNCAYAAYGC NAAPACNAAR CCNMGNGARG ARCARTTYAA
YWSNACNTAY MGNGTNGTNW SNGTNYTNAC NGTNYTNCAY
CARGAYTGGY TNAAYGGNAA RGARTAYAAR TGYAARGTNW
SNAAYAARGG NYTNCCNWSN WSNATHGARA APACNATHWS
NAARGCNAAR GGNCARCCNM GNGARCCNCA RGTNTAYACN
YTNCCNCCNW SNCARGARGA RATGACNAAR AAYCARGTNW
SNYTNACNTG YYTNGTNAAR GGNTTYTAYC CNWSNGAYAT
HGCNGTNGAR TGGGARWSNA AYGGNCARCC NGARAAYAAY
TAYAAPACNA CNCCNCCNGT NYTNGAYWSN GAYGGNWSNT
TYTTYYTNTA YWSNMGNYTN ACNGTNGAYA ARWSNMGNTG
GCARGARGGN AAYGTNTTYW SNTGYWSNGT NATGCAYGAR
GCNYTNCAYA AYCAYTAYAC NCAPAARWSN YTNWSNYTNW
SNYTNGGNAA R

In an embodiment, the polynucleotide encodes human interferon-alfa-2a-human IgG4 Fc comprising the following nucleotide sequence:

(SEQ ID NO: 5)

TGYGAYYTNC CNCARACNCA YWSNYTNGGN WSNMGNMGNA
CNYTNATGYT NYTNGCNCAR ATGMGNAARA THWSNYTNTT
YWSNTGYYTN AARGAYMGNC AYGAYTTYGG NTTYCCNCAR
GARGARTTYG GNAAYCARTT YCAPAARGCN GARACNATHC
CNGTNYTNCA YGARATGATH CARCARATHT TYAAYYTNTT
YWSNACNAAR GAYWSNWSNG CNGCNTGGGA YGAPACNYTN
YTNGAYAART TYTAYACNGA RYTNTAYCAR CARYTNAAYG
AYYTNGARGC NTGYGTNATH CARGGNGTNG GNGTNACNGA
RACNCCNYTN ATGAARGARG AYWSNATHYT NGCNGTNMGN
AARTAYTTYC ARMGNATHAC NYTNTAYYTN AARGARAARA
ARTAYWSNCC NTGYGCNTGG GARGTNGTNM GNGCNGARAT
HATGMGNWSN TTYWSNYTNW SNACNAAYYT NCARGARWSN
YTNMGNWSNA ARGARGCNWS NGAYAARACN CAYACNTGYC
CNCCNTGYCC NGCNCCNGAR TTYYTNGGNG GNCCNWSNGT
NTTYYTNTTY CCNCCNAARC CNAARGAYAC NYTNATGATH
WSNNGNACNC CNGARGTNAC NTGYGTNGTN GTNGAYGTNW
SNCARGARGA YCCNGARGTN CARTTYAAYT GGTAYGTNGA
YGGNGTNGAR GTNCAYAAYG CNAAPACNAA RCCNMGNGAR
GARCARTTYA AYWSNACNTA YMGNGTNGTN WSNGTNYTNA
CNGTNYTNCA YCARGAYTGG YTNAAYGGNA ARGARTAYAA
RTGYAARGTN WSNAAYAARG GNYTNCCNWS NWSNATNGAR
AARACNATHW SNAARGCNAA RGGNCARCCN MGNGARCCNC
ARGTNTAYAC NYTNCCNCCN WSNCARGARG ARATGACNAA
RAAYCARGTN WSNYTNACNT GYYTNGTNAA RGGNTTYTAY
CCNWSNGAYA THGCNGTNGA RTGGGARWSN AAYGGNCARC
CNGAPAAYAA YTAYAARACN ACNCCNCCNG TNYTNGAYWS
NGAYGGNWSN TTYTTYYTNT AYWSNMGNYT NACNGTNGAY
AARWSNNGNT GGCARGARGG NAAYGTNTTY WSNTGYWSNG
TNATGCAYGA RGCNYTNCAY AAYCAYTAYA CNCARAARWS
NYTNWSNYTN WSNYTNGGNA AR

In an embodiment, the polynucleotide encodes human interferon-alpha con-1-human IgG4 Fc comprising the following nucleotide sequence:

(SEQ ID NO: 16)

ATGTGYGAYY TNCCNCAPAC NCAYWSNYTN GGNAAYMGNM
GNGCNYTNAT HYTNYTNGCN CARATGMGNM GNATHWSNCC
NTTYWSNTGY YTNAARGAYM GNCAYGAYTT YGGNTTYCCN
CARGARGART TYGAYGGNAA YCARTTYCAR AARGCNCARG
CNATHWSNGT NYTNCAYGAR ATGATHCARC APACNTTYAA
YYTNTTYWSN ACNAARGAYW SNWSNGCNGC NTGGGAYGAR
WSNYTNYTNG ARAARTTYTA YACNGARYTN TAYCARCARY
TNAAYGAYYT NGARGCNTGY GTNATHCARG ARGTNGGNGT
NGARGARACN CCNYTNATGA AYGTNGAYWS NATHYTNGCN
GTNAAPAART AYTTYCARMG NATHACNYTN TAYYTNACNG
ARAARAARTA YWSNCCNTGY GCNTGGGARG TNGTNNGNGC
NGARATHATG MGNWSNTTYW SNYTNWSNAC NAAYYTNCAR
GARMGNYTNM GNMGNAARGA RGCNWSNGAY AARACNCAYA
CNTGYCCNCC NTGYCCNGCN CCNGARTTYY TNGGNGGNCC
NWSNGTNTTY YTNTTYCCNC CNAARCCNAA RGAYACNYTN
ATGATHWSNM GNACNCCNGA RGTNACNTGY GTNGTNGTNG
AYGTNWSNCA RGARGAYCCN GARGTNCART TYAAYTGGTA
YGTNGAYGGN GTNGARGTNC AYAAYGCNAA RACNAARCCN
MGNGARGARC ARTTYAAYWS NACNTAYMGN GTNGTNWSNG
TNYTNACNGT NYTNCAYCAR GAYTGGYTNA AYGGNAARGA
RTAYAARTGY AARGTNWSNA AYAARGGNYT NCCNWSNWSN
ATHGARAARA CNATHWSNAA RGCNAARGGN CARCCNMGNG
ARCCNCARGT NTAYACNYTN CCNCCNWSNC ARGARGAPAT
GACNAAPAAY CARGTNWSNY TNACNTGYYT NGTNAARGGN
TTYTAYCCNW SNGAYATHGC NGTNGARTGG GARWSNAAYG
GNCARCCNGA RAAYAAYTAY AAPACNACNC CNCCNGTNYT
NGAYWSNGAY GGNWSNTTYT TYYTNTAYWS NNGNYTNACN
GTNGAYAARW SNMGNTGGCA RGARGGNAAY GTNTTYWSNT
GYWSNGTNAT GCAYGARGCN YTNCAYAAYC AYTAYACNCA
RAARWSNYTN WSNYTNWSNY TNGGNAAR

The present invention further includes any fusion polypeptide comprising IgG4 fused to a short half-life cytokine such as IL-10 (IL-10-IgG4 fusion). In an embodiment of the invention, human IL-10 comprises the amino acid sequence:

MHSSALLCCL VLLTGVPASP GQGTQSENSC THFPGNLPNM
LRDLRDAFSR VKTFFQMKDQ LDNLLLKESL LEDFKGYLGC
QALSEMIQFY LEEVMPQAEN QDPDIKAHVN SLGENLKTLR
LRLRRCHRFL PCENKSKAVE QVKNAFNKLQ EKGIYKANSE
FDIFINYIEA YMTMKIRN
(SEQ ID NO: 24; see also Genbank accession nos.:
CAH71813; AAV38450; AAX36831; CAG46825 or
XP_525040).

In an embodiment of the invention, the human IL-10-IgG4 fusion comprises the amino acid sequence:

(SEQ ID NO: 25)

MHSSALLCCLVLLTGVRASPGQGTQSENSCTHGPGNLPNMLRDLRDAFSR
VKTFFQMKDQLDNLLLKESLLEDFKGYLGCQALSEMIQFYLEEVMPQAEN
QDPDIKAHVNSLGENLKTLRLRLRRCHRFLPCENKSKAVEQVKNAFNKLQ
EKGIYKAMSEFDIFINYIEAYMTMKIRNASDKTHTCPPCPAPEFLGGPSV
FLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK

wherein, in an embodiment of the invention, the N-terminus of the fusion optionally comprises a methionine (M); wherein the linker is underscored and is also optional; optionally comprising a signal sequence; wherein the sequence that is N-terminal to the linker is human IL-10 and the sequence that is C-terminal to the linker is human IgG4 Fc. In an embodiment, the linker amino acid sequence is selected from SEQ ID NOs: 7-11 and 17-20.

### Therapeutic Methods

The present invention provides compositions and methods for treating or preventing any condition that is alleviated by administration of an interferon. For example, the present invention comprises method for treating or preventing an infection, in a host, subject or patient, by a virus which is a member of the Flaviviridae family in a host by administering a therapeutically effective dosage of an IFN-IgG4 fusion of the present invention, or a pharmaceutical composition thereof; optionally in association with any further therapeutic agent set forth below under the “Pharmaceutical Compositions” section (e.g., ribavirin), to the host, subject or patient. For example, the present invention includes, but is not limited to methods for treating or preventing infections caused by members of the Hepacivirus genus which includes the hepatitis-C virus (HCV). HCV includes several types, subtypes and isolates:
hepatitis C virus (isolate 1)
hepatitis C virus (isolate BK)
hepatitis C virus (isolate EC1)
hepatitis C virus (isolate EC10)
hepatitis C virus (isolate HC-J2)
hepatitis C virus (isolate HC-J5)
hepatitis C virus (isolate HC-J6)
hepatitis C virus (isolate HC-J7)
hepatitis C virus (isolate HC-J8)
hepatitis C virus (isolate HC-JT)
hepatitis C virus (isolate HCT18)
hepatitis C virus (isolate HCT27)
hepatitis C virus (isolate HCV-476)
hepatitis C virus (isolate HCV-KF)
hepatitis C virus (isolate Hunan)
hepatitis C virus (isolate Japanese)
hepatitis C virus (isolate Taiwan)
hepatitis C virus (isolate TH)
hepatitis C virus isolate H
hepatitis C virus type 1
   hepatitis C virus type 1a
      hepatitis C virus strain H77
   hepatitis C virus type 1b
   hepatitis C virus type 1c
   hepatitis C virus type 1d
   hepatitis C virus type 1e
   hepatitis C virus type 1f
hepatitis C virus type 10
hepatitis C virus type 2
   hepatitis C virus type 2a
   hepatitis C virus type 2b
   hepatitis C virus type 2c
   hepatitis C virus type 2d
   hepatitis C virus type 2f
hepatitis C virus type 3
   hepatitis C virus type 3a
   hepatitis C virus type 3b
   hepatitis C virus type 3g
hepatitis C virus type 4
   hepatitis C virus type 4a
   hepatitis C virus type 4c
   hepatitis C virus type 4d
   hepatitis C virus type 4f
   hepatitis C virus type 4h
   hepatitis C virus type 4k
hepatitis C virus type 5
   hepatitis C virus type 5a
hepatitis C virus type 6
   hepatitis C virus type 6a
hepatitis C virus type 7
   hepatitis C virus type 7a
   hepatitis C virus type 7b
hepatitis C virus type 8
   hepatitis C virus type 8a

The present invention also includes methods and compositions for treating or preventing infection caused by members of the Flavivirus genus. The Flavivirus genus includes Yellow fever virus; Tick-borne viruses such as the Gadgets Gully virus, Kadam virus, Kyasanur Forest disease virus, Langat virus, Omsk hemorrhagic fever virus, Powassan virus, Royal Farm virus, Karshi virus, Tick-borne encephalitis virus, Neudoerfl virus, Sofjin virus, Louping ill virus and the Negishi virus; seabird tick-borne viruses such as the Meaban virus, Saumarez Reef virus, and the Tyuleniy virus; mosquito-borne viruses such as the Aroa virus, Bussuquara virus, Iguape virus and the Naranjal virus; Dengue viruses such as the Dengue virus and the Kedougou virus; Japanese encephalitis viruses such as the Cacipacore virus, Koutango virus, Japanese encephalitis virus, Murray Valley encephalitis virus, Alfuy virus, St. Louis encephalitis virus, Usutu virus, West Nile virus, Kunjin virus and the Yaounde virus; Kokobera viruses such as the Kokobera virus and the Stratford virus; Ntaya viruses such as the Bagaza virus, Ilheus virus, Rocio virus, Israel turkey meningoencephalomyelitis virus, Ntaya virus and the Tembusu virus; Spondweni viruses such as the Zika virus and the Spondweni virus; Yellow fever viruses such as the Banzi virus, Bouboui virus, Edge Hill virus, Jugra virus, Saboya virus, Potiskum virus, Sepik virus, Uganda S virus, Wesselsbron virus and the Yellow fever virus; Entebbe viruses such as the Entebbe bat virus, Sokoluk virus, and the Yokose virus; Modoc viruses such as the Apoi virus, Cowbone Ridge virus, Jutiapa virus, Modoc virus, Sal Vieja virus and the San Perlita virus; Rio Bravo viruses such as the Bukalasa bat virus, Carey Island virus, Dakar bat virus, Montana myotis leukoencephalitis virus, Phnom Penh bat virus, Batu Cave virus, Rio Bravo virus, Tamana bat virus, and the Cell fusing agent virus.

The present invention includes methods and compositions for treating or preventing infection caused by members of the Pestivirus genus. The Pestivirus genus includes, Border disease virus (sheep), Bovine viral diarrhea virus 1, Bovine viral diarrhea virus 2, Classical swine fever virus, and Hog cholera virus.

Moreover, the present invention includes methods and compositions for treating or preventing infections caused by Hepatitis G virus or Hepatitis GB virus-A, B or C.

In an embodiment, the subject is administered the IFN-IgG4 fusion of the invention, optionally, in association with a further therapeutic agent; for example a therapeutically effective amount another anti-viral therapy, for a treatment time period sufficient to eradicate detectable hepatitic C virus-RNA and to maintain no detectable hepatitic C virus RNA for at least twelve weeks (e.g., 24 weeks) after the end of the treatment time period.

As also discussed below, the term “in association” indicates that an IFN-IgG4 fusion and the further therapeutic agent (e.g., anti-viral therapy) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., orally, intravenously, subcutaneously).

A “host”, “subject” or “patient” may be any organism, such as a mammal (e.g., primate, chimpanzee, dog (e.g., beagle), cat, cow, horse, pig, goat, rabbit, rat (e.g., Sprague Dawley rat), mouse, bird), for example a human. Accordingly, methods of the invention include embodiments wherein fusions of the invention are used to treat a human, for example, in a clinical setting or to treat an animal, for example, by a veterinarian or by a researcher (e.g., performing toxicology, pharmacokinetics or safety assessment studies). In an embodiment, the “host”, “subject” or “patient” is pregnant or a nursing mother.

A person suffering from chronic hepatitis C infection may exhibit one or more of the following signs or symptoms:
(a) elevated ALT,
(b) positive test for anti-HCV antibodies,
(c) presence of HCV as demonstrated by a positive test for HCV-RNA,
(d) clinical stigmata of chronic liver disease,
(e) hepatocelluar damage.

Such criteria may not only be used to diagnose hepatitis C viral infection, but can be used to follow and evaluate a patient's response to drug treatment. Such parameters may be used, by a clinician, to modulate the dose and duration of treatment. Evaluation of such criteria and adjustment of a host, patient or subject's treatment regimen can be performed easily by an practitioner of ordinary skill in the art.

The present inventions methods and compositions can be used in a liver transplantation procedure to treat or prevent Flaviviridae infection in the recipient. The donor liver can come from a living donor (i.e., living donor liver transplantation (LDLT)) wherein a portion of the donor's liver is removed and introduced into the recipient. Alternatively, the transplant can be from a deceased donor wherein the entire liver is removed and transplanted. For example, an embodiment of the invention comprises a method for preventing infection of a host, with a virus which is a member of the Flaviviridae family of viruses, following transplantation of a liver into said host or transfusion of blood into said host comprising administering to said host a therapeutically effective amount of IFN-IgG4 or a pharmaceutically acceptable composition thereof.

The present invention also comprises a method for treating or preventing multiple sclerosis or a relapse of multiple sclerosis in a subject comprising administering to the subject a therapeutically effective amount of IFN-IgG4 or a pharmaceutically acceptable composition thereof. For example, in an embodiment, the subject is administered IFN beta-1a-IgG4 or IFN beta-1b-IgG4. Optionally, IFN-IgG4 is administered in association with one or more of Tolterodine; Oxybutynin; Oxybutynin; Oxybutynin; Propantheline bromide; Trospium chloride; Imipramine; Solifenacin succinate; Mineral oil; Docusate; Bisacodyl; Docusate stool softener laxative; Sodium phosphate; Psyllium hydrophilic mucilloid; Magnesium hydroxide; Glycerin; Glatiramer acetate; Mitoxantrone; Duloxetine hydrochloride; Venlafaxine; Paroxetine; Fluoxetine; Bupropion; Sertraline; Meclizine; Papaverine; Tadalafil; Vardenafil; Alprostadil; Alprostadil; Sildenafil; Dexamethasone; Prednisone; Methylprednisolone; Amantadine; Modafinil; Fluoxetine; Pemoline; Hydroxyzine; Meclizine; Duloxetine hydrochloride; Phenytoin; Amitriptyline; Gabapentin; Nortriptyline; Clonazepam; Carbamazepine; Imipramine; Baclofen; Dantrolene; Baclofen (intrathecal); Clonazepam; Diazepam; Tizanidine; Isoniazid; Clonazepam; Desmopressin; Desmopressin; Sulfamethoxazole; Ciprofloxacin; Methenamine; Nitrofurantoin; or Phenazopyridine or a pharmaceutical composition thereof.

The present invention also comprises method for treating (e.g., reducing the severity or erradicating) or preventing serious infections associated with chronic granulomatous disease in a subject comprising administering to the subject a therapeutically effective amount of IFN-IgG4 or a pharmaceutically acceptable composition thereof. For example, in an embodiment, the subject is administered IFN gamma-1b-IgG4. Optionally, IFN-IgG4 is administered in association with one or more of a combination of trimethoprim and sulfamethazole or trimethoprim or itraconizole or a pharmaceutical composition thereof.

The present invention also comprises method for treating or preventing or delaying time to disease progression, in patients with severe, malignant osteopetrosis, comprising administering, to the patient, a therapeutically effective amount of IFN-IgG4 or a pharmaceutically acceptable composition thereof. For example, in an embodiment, the subject is administered IFN gamma-1b-IgG4. Optionally, IFN-IgG4 is administered in association with one or more of calcitriol or prednisone or a pharmaceutical composition thereof.

The present invention also comprises method for treating or preventing refractory or recurring external condylomata acuminata, comprising administering (e.g., by intralesional injection), to the patient, a therapeutically effective amount of IFN-IgG4 or a pharmaceutically acceptable composition thereof. For example, in an embodiment, the subject is administered interferon alfa-n3-IgG4. Optionally, IFN-IgG4 is administered in association with one or more of trichloroacetic acid, podophyllum, topical liquid nitrogen treatment, podophyllo-toxin paint, imiquimod cream, podofilox solution, 5-fluorouracil cream or trichloroacetic acid (TCA) or a pharmaceutical composition thereof.

The present invention also comprises method for treating or preventing, in a subject, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, chronic hepatitis B comprising administering, to the subject, a therapeutically effective amount of IFN-IgG4 or a pharmaceutically acceptable composition thereof. For example, in an embodiment, the subject is administered interferon alfa-2a-IgG4 or interferon alfa-2b-IgG4. Optionally, IFN-IgG4 is administered in association with one or more of cladribine (2-chlorodeoxyadenosine, 2-CdA), pentostatin, imatinib, podophyllum, topical liquid nitrogen treatment, podophyllo-toxin paint, imiquimod cream, podofilox solution, 5-fluorouracil cream, trichloroacetic acid (TCA), rituximab, tositumomab and iodine I¹³¹, Ibritumomab tiuxetan, dacarbazine, aldesleukin or doxorubicin hydrochloride or a pharmaceutical composition thereof.

The present invention includes methods for treating or preventing any inflammatory disorder (e.g., multiple sclerosis, inflammatory bowel syndrome, psoriasis, Crohn's disease, rheumatoid arthritis, or ulcerative colitis) in a subject, by administering, to the subject, a therapeutically effective amount of an IL-10-IgG4 fusion.

In an embodiment of the invention, an IFN-IgG4 fusion of the invention is administered to a patient, subject or host, in any of the foregoing methods, that is pregnant or a nursing mother, because of reduced fetal toxicity and toxicity to nursing infants, whose mother is administered IFN-IgG4, as compared to other IFN fusions. Without being bound by a single theory or mechanism of action, the IFN-IgG4 fusions of the invention may exhibit lower fetal toxicity and toxicity to nursing infants due to the presence of the IgG4 moiety which exhibits limited placental transfer.

### Pharmaceutical Compositions

The present invention includes methods for using a pharmaceutical composition comprising an IFN-IgG4 fusion, optionally in association with a further therapeutic agent, and a pharmaceutically acceptable carrier for treating a Flaviviridae infection along with the pharmaceutical compositions themselves. The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

A pharmaceutical composition containing an IFN-IgG4 fusion can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular) and non-parenteral (e.g., oral, transdermal, intranasal, intraocular, sublingual, inhalation, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations are generally added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

Implantation of a slow-release or sustained-release system, such that a constant level of dosage is maintained is also contemplated herein. Briefly, in this embodiment, a IFN-IgG4 fusion is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient diffuses through the outer polymeric membrane in a release rate controlling step. The percentage of active compound contained in such parenteral compositions is highly dependent on the specific nature thereof, as well as the activity of the IFN-IgG4 fusion and the needs of the subject.

The concentration of the IFN-IgG4 fusion can be adjusted so that an injection provides an effective amount to produce the desired pharmacological effect. The exact dose depends, inter alia, on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

IFN-IgG4 fusion can be formulated into a lyophilized powder, which can be reconstituted for administration as solutions, emulsions and other mixtures. The powder may also be reconstituted and formulated as a solid or gel.

The sterile, lyophilized powder is prepared by dissolving IFN-IgG4 fusion, or a pharmaceutically acceptable derivative thereof, in a suitable solvent. The solvent may contain an excipient which improves the stability or another pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial can contain a single dosage or multiple dosages of the IFN-IgG4. The lyophilized powder can be stored under appropriate conditions, such as at about 4° C. to room temperature.

Reconstitution of this lyophilized powder with water for injection provides a formulation for use in parenteral administration. For reconstitution, the lyophilized powder can be added to sterile water or another suitable carrier. The precise amount depends upon the selected compound. Such amount can be empirically determined.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing an IFN-IgG4 fusion, by itself or associated with an excipient, in powder form.

In an embodiment, IFN-IgG4 fusion is formulated into a solid dosage form for oral administration, in one embodiment, into a capsule or tablet. Tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

Methods of the present invention include administration of an IFN-IgG4 fusion in association with, for example, one or more other therapeutic agents. In an embodiment, the other therapeutic agent is an anti-viral agent that, when administered to a subject, treats or prevents a viral infection in the subject. The administration and dosage of such agents is typically as according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

A “therapeutic agent” is an agent that, when administered to a subject brings about a desired or beneficial therapeutic effect, such as prevention, elimination or reduction of the progression or severity of symptoms associated with a given medical condition. A therapeutic agent may be, for example, an anti-viral agent or an anti-cancer agent.

Compounds that may be administered or combined in association with an IFN-IgG4 fusion include one or more ribonucleoside analogues, IMPDH inhibitors, N-glycosylation inhibitors, N3 protease inhibitors, NS5B inhibitors, immunomodulatory compounds and CTP synthase inhibitors, thiazolidine derivatives, benzanilides, phenanthrenequinones, helicase inhibitors, polymerase inhibitors, antisense phosphothioate oligodeoxynucleotides, IRES-dependent translation inhibitors, nuclease resistant ribozymes, 1-amino-alkyloyclohexanes, alkyl lipids, antioxidants, squalene, amantadine, bile acids, N-(phosphonoacetyl)-L-aspartic acid, benzenedicarboxamides, polyadenylic acid derivatives, 2′,3′ dideoxyinosine and benzimidazoles.

As mentioned above, in an embodiment of the present invention, ribavirin 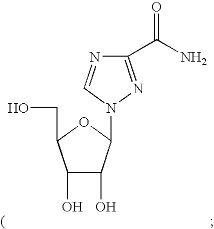
1-β-D-ribofuranosyl-1H-1,2,4-triazole-3-carboxamide) is administered or combined in association with an IFN-IgG4 fusion. Ribavirin is sold as REBETOL® by Schering Corporation; Kenilworth, N.J. Its manufacture and formulation is described, for example, in U.S. Pat. No. 4,211,771.

As mentioned above, in an embodiment of the present invention, lamuvidine 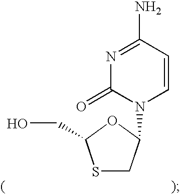
or zidovudine 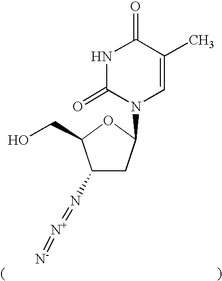
is administered or combined in association with an IFN-IgG4 fusion.

In another embodiment of the invention, gemcitabine 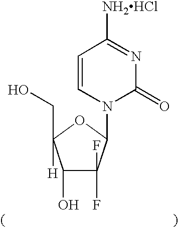
is administered or combined in association with an IFN-IgG4 fusion. Gemcitabine is sold as GEMZAR® by Eli Lilly and Co. (Indianapolis, Ind.).

A further embodiment of the present invention comprises administration of or a combination of an IFN-IgG4 fusion in association with VX497 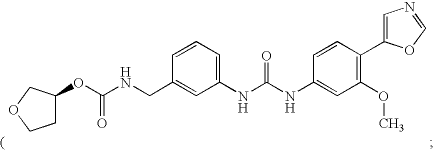
Vertex Pharmaceuticals; Cambridge, Mass.).

An embodiment of the invention comprises administration of or a combination of mycophenolate mofetil (MMF; 2-morpholinoethyl (E)-6-(1,3-dihydro-4-hydroxy-6-methoxy-7-methyl-3-oxo-5-isobenzofuranyl)-4-methyl-4-hexenoate) in association with an IFN-IgG4 fusion. MMF is sold as CellCept® by Roche Laboratories (Nutley, N.J.).

Another embodiment comprises administration of or a combination of EICAR 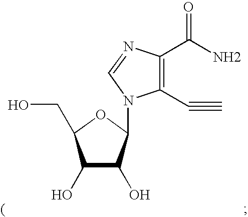
5-ethynyl-1-beta-D-ribofuranosylimidazole-4-carboxamide; Balzarini et al., J. Biol. Chem. 268(33): 24591-24598 (1993)) or 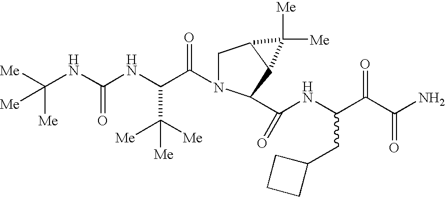
in association with an IFN-IgG4 fusion.

An embodiment of the present invention comprises administration of or a combination of tiazofurin 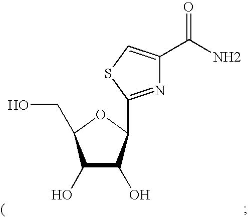
Balzarini et al., J. Biol. Chem. 268(33): 24591-24598 (1993)) in association with an IFN-IgG4 fusion.

Another embodiment of the invention comprises administration of or a combination of deoxynojirimycin and/or derivatives thereof, such as N-nonyl-deoxynojirimycin (De Clercq et al., Mini Rev Med Chem. 2(2):163-75 (2002)) or n-butyl deoxynojirimycin (nB-DNJ; Ouzounov et al., Antiviral Res. 55(3):425-35 (2002)), in association with an IFN-IgG4 fusion.

In another embodiment, BILN-2061 ( 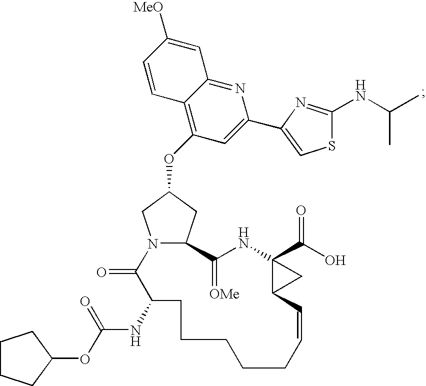
Lamarre et al., Nature 426(6963):129-31 (2003)), is administered or combined in association with an IFN-IgG4 fusion.

In another embodiment, thymalfasin (e.g., ZADAXIN™) is administered or combined in association with an IFN-IgG4 fusion. ZADAXIN™ is available from SciClone Pharmaceuticals International, Ltd. (San Mateo, Calif.).

In yet another embodiment, isatoribine 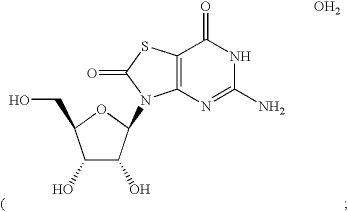
ANA245; 5-Amino-3-beta-D-ribofuranosylthiazolo(4,5-d)pyrimidine-2,7(3H,6H)-dione monohydrate; Thiazolo(4,5-d)pyrimidine-2,7(3H,4H)-dione, 5-amino-3-beta-D-ribofuranosyl-, monohydrate) is administered or combined in association with an IFN-IgG4 fusion.

In another embodiment, an IFN-IgG4 fusion is administered or combined in association with any NS5B inhibitor such as telbivudine 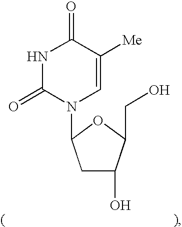
valtorcitibine 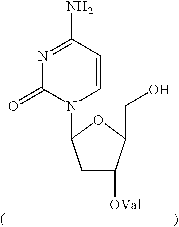
MN283 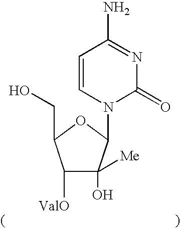
or NM107 (Idenix Pharmaceuticals; Cambridge, Mass.).

In another embodiment, an IFN-IgG4 fusion is administered or combined in association with 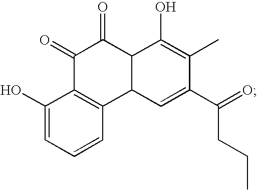
Chu et al., Tetrahedron Letters 37(40): 7229-7232 (1996)) or 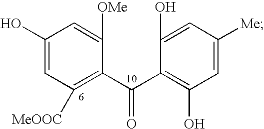
Biorg. Med. Chem. Lett. 9(14): 1949-1952 (1999)).

In a further embodiment, an IFN-IgG4 fusion is administered or combined in association with any of the P₁ variants of Elgin c disclosed in Qasim et al., Biochemistry 36: 1598-1607 (1997).

In yet another embodiment, an IFN-IgG4 fusion is administered or combined in association with gliotoxin 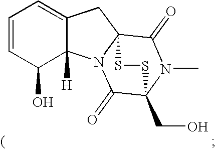
Ferrari et al., J. Virology 73(2): 1649-1654 (1999)).

Other embodiments of the invention include administration of or a combination of an IFN-IgG4 fusion in association with RD3-4082 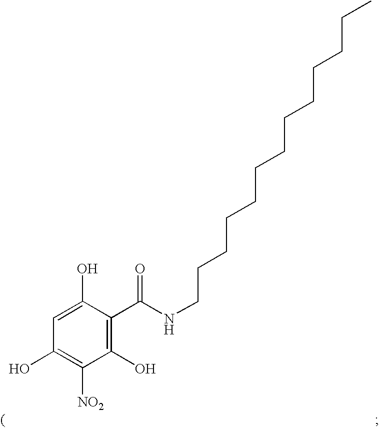
Sudo et al., Anti-viral Chem. & Chemother. 9: 186 (1998)) or with RD3-4078 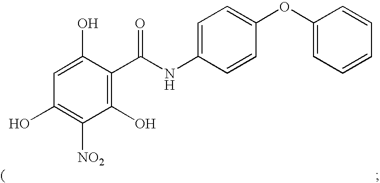
Sudo et al., Anti-viral Chem. & Chemother. 9: 186 (1998)) or any other protease inhibitor disclosed in Sudo et al.

A further embodiment of the invention comprises administration of or a combination of an IFN-IgG4 fusion in association with 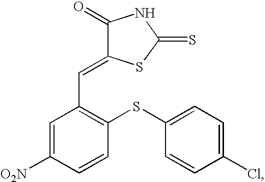 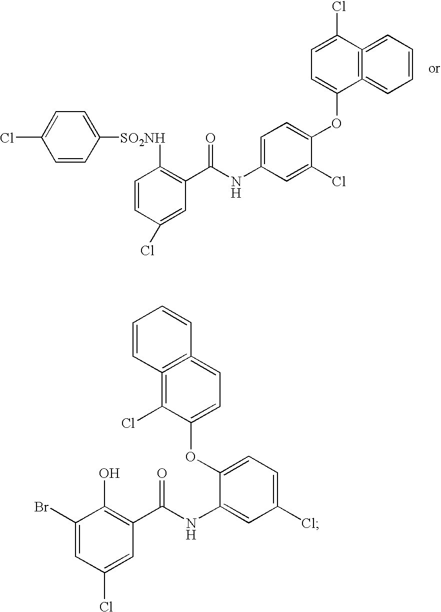
Kakiuchi et al., FEBS Letters 421: 217-220 (1998)) or any other proteinase inhibitor disclosed in Kakiuchi et al.

Another embodiment of the present invention comprises administration of or a combination of an IFN-IgG4 fusion in association with RD4-6205 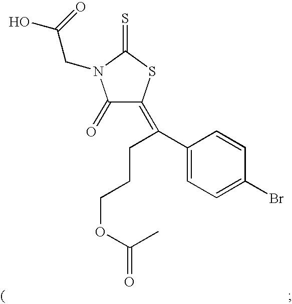
Sudo et al., Biochem. Biophys. Res. Comm. 238: 643-647 (1997)) or any other protease inhibitor disclosed in Sudo et al.

An embodiment of the present invention comprises administration of or a combination of an IFN-IgG4 fusion in association with cerulenin 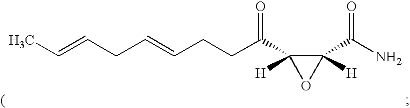
CAS Registry No. 17397-89-6; Lohmann et al., Virology 249: 108-118 (1998)) or any other HCV RNA-dependent RNA polymerase (RdRp) inhibitor disclosed in Lohmann et al.

An embodiment of the present invention comprises administration of or a combination of an IFN-IgG4 fusion in association with ceplene 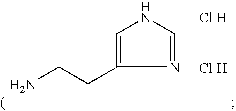
2-(1H-Imidazol-4-yl)ethanamine dihydrochloride).

Yet another embodiment of the present invention comprises administration of or a combination of an IFN-IgG4 fusion in association with amantadine

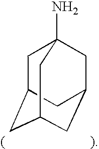

A further embodiment of the present invention comprises administration of or a combination of an IFN-IgG4 fusion in association with IDN-6556 (

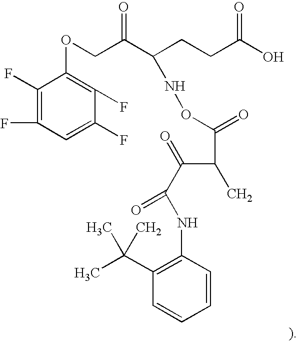

Yet another embodiment of the present invention comprises administration of or a combination of an IFN-IgG4 fusion in association with naphthoquinone, 2-methylnaphthoquinone, 2-hydroxynaphthoquinone, 5-hydroxynaphthoquinone, 5,8-dihydroxynaphthoquinone, alkannin or shikonin (Takeshita et al., Analytical Biochem. 247: 242-246 (1997)).

A further embodiment of the present invention comprises administration of or a combination of an IFN-IgG4 fusion in association with 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane, 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethylcyclohexane, or N-(1,3,3,5,5-pentamethylcyclohexyl) pyrrolidine or any other 1-aminoalkylcyclohexane N-methyl-D-aspartate (NMDA) inhibitors disclosed in U.S. Pat. No. 6,034,134.

A further embodiment of the present invention comprises administration of or a combination of an IFN-IgG4 fusion in association with d-α-tocopherol or any other anti-HCV compound disclosed in U.S. Pat. No. 5,922,757.

Another embodiment of the present invention comprises administration of or a combination of an IFN-IgG4 fusion in association with tauroursodeoxycholic acid, chenodeoxycholic acid, ursodeoxycholic acid or free bile acid or any other bile acid HCV inhibitor disclosed in U.S. Pat. No. 5,846,964.

Another embodiment of the present invention comprises administration of or a combination of an IFN-IgG4 fusion in association with 1,1′-[1,4-phenylenebis (methylene)]bis(4,4′-trans-(4,5,6,7,8,9-hexahydro) benzimidazoyl)piperidine, 1,1′-[1,4-phenylenebis(methylene)]bis(4,4′-benzimidazoyl) piperidine or any other anti-HCV compound disclosed in U.S. Pat. No. 5,830,905.

Another embodiment of the present invention comprises administration of or a combination of an IFN-IgG4 fusion in association with N,N′-4-[(2-benzimidazole)phenyl]-1,4-butanedicarboxamide, N,N′-4-[(2-benzimidazole)phenyl]-1,6-hexanedicarboxamide, N,N′-4-[(2-benzimidazole)phenyl]-1,8-octanedicarboxamide, N,N′-4-[(2-benzimidazole)phenyl]-1,9-nonanedicarboxamide, N,N′-4-[(2-benzimidazole)phenyl]-1,10-decanedicarboxamide or N,N′-4-[(2-benzimidazole)phenyl]-1,4-butenedicarboxamide or any other carboxamide HCV inhibitor disclosed in U.S. Pat. No. 5,633,388.

Another embodiment of the present invention comprises administration of or a combination of an IFN-IgG4 fusion in association with any of the polyadenylic acid (5′) derivatives disclosed in U.S. Pat. No. 5,496,546.

A further embodiment of the present invention comprises administration of or a combination of an IFN-IgG4 fusion in association with 2′,3′-dideoxyinosine (U.S. Pat. No. 5,026,687).

An embodiment of the present invention comprises administration of or a combination of an IFN-IgG4 fusion in association with 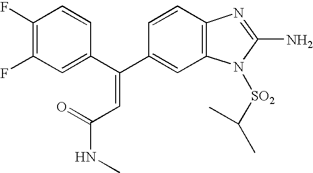
or any other benzimidazole disclosed in U.S. Pat. No. 5,891,874.

An additional embodiment of the invention comprises administration of or a combination of VX-950 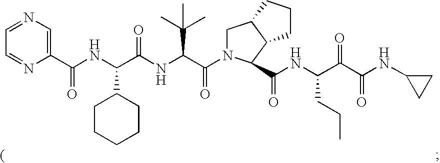
Lin et al., J. Biol. Chem. 279(17): 17508-17514 (2004)) in association with an IFN-IgG4 fusion.

Another embodiment of the present invention comprises administration of or a combination of an IFN-IgG4 fusion in association with viramidine 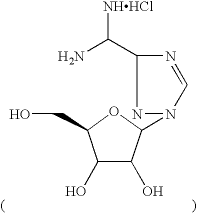
or levovirin

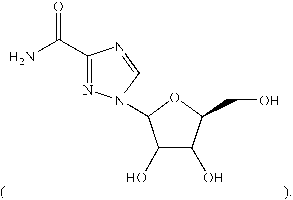

In an embodiment, the present invention comprises administration of or a combination of IFN-IgG4 in association with one or more selected from: Tolterodine; Oxybutynin; Oxybutynin; Oxybutynin; Propantheline bromide; Trospium chloride; Imipramine; Solifenacin succinate; Mineral oil; Docusate; Bisacodyl; Docusate stool softener laxative; Sodium phosphate; Psyllium hydrophilic mucilloid; Magnesium hydroxide; Glycerin; Glatiramer acetate; Mitoxantrone; Duloxetine hydrochloride; Venlafaxine; Paroxetine; Fluoxetine; Bupropion; Sertraline; Meclizine; Papaverine; Tadalafil; Vardenafil; Alprostadil; Alprostadil; Sildenafil; Dexamethasone; Prednisone; Methylprednisolone; Amantadine; Modafinil; Fluoxetine; Pemoline; Hydroxyzine; Meclizine; Duloxetine hydrochloride; Phenytoin; Amitriptyline; Gabapentin; Nortriptyline; Clonazepam; Carbamazepine; Imipramine; Baclofen; Dantrolene; Baclofen; Clonazepam; Diazepam; Tizanidine; Isoniazid; Clonazepam; Desmopressin; Desmopressin; Sulfamethoxazole; Ciprofloxacin; Methenamine; Nitrofurantoin; Phenazopyridine, a combination of trimethoprim and sulfamethazole or trimethoprim, itraconizole, calcitriol, prednisone, trichloroacetic acid, podophyllum, topical liquid nitrogen treatment, podophyllo-toxin paint, imiquimod cream, podofilox solution, 5-fluorouracil cream, trichloroacetic acid (TCA), cladribine (2-chlorodeoxy adenosine, 2-CdA), pentostatin, imatinib, of trichloroacetic acid, podophyllum, topical liquid nitrogen treatment, podophyllo-toxin paint, imiquimod cream, podofilox solution, 5-fluorouracil cream, trichloroacetic acid (TCA), rituximab, tositumomab and iodine I¹³¹, Ibritumomab tiuxetan, dacarbazine, aldesleukin or doxorubicin hydrochloride.

As also discussed above, compositions and methods of the invention include an IFN-IgG4 fusion optionally “in association” with one or more additional anti-viral agents (e.g., ribavirin, interferon alfa-2a or 2b, or pegylated interferon alfa-2a or 2b). The term “in association” indicates that the components of the combinations of the invention can be formulated into a single composition for simultaneous delivery or formulated separately into two or more compositions (e.g., a kit). Furthermore, each component of a combination of the invention can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., orally, intravenously, subcutaneously).

The present invention further comprises compositions comprising IFN-IgG4 fusion in association with one or more anti-viral agents discussed above (e.g., ribavirin) along with pharmaceutical compositions thereof comprising a pharmaceutically acceptable carrier.

### Dosage and Administration

Typical protocols for the therapeutic administration of such substances are well known in the art. Pharmaceutical composition of the invention may be administered, for example, by any parenteral (e.g., subcutaneous injection, intramuscular injection, intravenous injection) or non-parenteral route (e.g., orally, nasally).

Pills and capsules of the invention can be administered orally. Injectable compositions can be administered with medical devices known in the art; for example, by injection with a hypodermic needle including the REDIPEN® or the NOVOLET® Novo Pen discussed above.

Injectable pharmaceutical compositions of the invention may also be administered with a needleless hypodermic injection device; such as the devices disclosed in U.S. Pat. Nos. 5,399,163; 5,383,851; 5,312,335; 5,064,413; 4,941,880; 4,790,824 or 4,596,556.

Compositions of the invention can be administered, for example, three times a day, twice a day, once a day, three times weekly, twice weekly or once weekly, once every two weeks or 3, 4, 5, 6, 7 or 8 weeks.

In an embodiment, the daily dose of a compound of the present invention or of any other anti-viral agent administered in association with a compound of the invention is, where possible, administered accordance with the Physicians' Desk Reference 2003 (Physicians' Desk Reference, 57th Ed); Medical Economics Company; ISBN: 1563634457; 57th edition (November 2002). The proper dosage can, however, be altered by a clinician to compensate for particular characteristics of the subject receiving the therapy depending, for example, on the potency of the compound administered, side-effects, age, weight, medical condition, overall health and response.

The term “therapeutically effective amount” means that amount of a therapeutic agent or substance (e.g., IFN-IgG4 fusion) that will elicit a biological or medical response of a tissue, system, subject or host that is being sought by the administrator (such as a researcher, doctor or veterinarian) which includes, for example, alleviation of the symptoms of Flaviviridae virus (e.g., HCV) infection and the prevention, slowing or halting of progression of Flaviviridae virus (e.g., HCV) infection and its symptom(s) to any degree including prevention of the infection of a host with a Flaviviridae virus (e.g., HCV) following transplant of a liver into said host; or, in an embodiment, which includes alleviation of the symptoms of multiple sclerosis, hepatitis B virus infection, condylomata acuminate, cancer (e.g., leukemia, lymphoma, melanoma, kaposi's sarcoma) or any medical disorder discussed herein and the prevention, slowing or halting of progression of such a medical disorder and its symptom(s) to any degree. For example, in one embodiment, a “therapeutically effective dosage” of IFN-IgG4 fusion or a combination including another anti-viral agent (e.g., ribavirin and/or pegylated or unpegylated inferferon alfa-2a or 2b) results in the eradication of detectable Flaviviridae Viral RNA (e.g., HCV RNA) for any period of time, for example, 12 or more weeks (e.g., 24 weeks). Detection of viral RNA in a host can be done easily using conventional well-known methods in the art.

In an embodiment, a therapeutically effective dosage or amount of any IFN-IgG4 fusion or IL-10-IgG4 fusion of the invention is about 2 mg/60 kg body weight to about 3 mg/60 kg body weight (e.g., about 2.1, 2.2, 2.3, 2.4, 2.5, 2.6, 2.7, 2.8 or 2.9 mg /60 kg body weight) with a dosing frequency of about once monthly to once every two months.

In an embodiment, a therapeutically effective dose of ribavirin (e.g., REBETROL®) depends on the patient's body weight. In an embodiment, the recommended dose of REBETOL® is provided, below, in Table 1

TABLE 1
Recommended Dosing

| Body weight | REBETOL Capsules |
|---|---|
| </=75 kg | 2 × 200-mg capsules AM, |
| | 3 × 200-mg capsules PM daily p.o. |
| >75 kg | 3 × 200 mg capsules AM, |
| | 3 × 200 mg capsules PM daily p.o. |

In an embodiment, the duration of treatment with ribavirin (e.g., REBETOL®) for patients previously untreated with interferon is 24 to 48 weeks. The duration of treatment should be individualized to the patient depending on baseline disease characteristics, response to therapy, and tolerability of the regimen. After 24 weeks of treatment, virologic response should be assessed. Treatment discontinuation can be considered in any patient who has not achieved an HCV RNA below the limit of detection of the assay by 24 weeks.

In an embodiment, in patients who relapse following interferon therapy, the duration of treatment with ribavirin (e.g., REBETOL®) is 24 weeks.

REBETOL® may be administered without regard to food, but should be administered in a consistent manner with respect to food intake.

A clinician or physician can adjust dosage of an IFN-Ig fusion of the invention according to the progress observed in the patient receiving the therapy. For example, the viral load of a patient suffering from a hepatitis virus (e.g., HCV) infection can be monitored using any of the many well known methods known in the art. In an embodiment of the invention, viral load is monitored by rtPCR or ELISA as discussed in more detail below (see e.g., Fabrizi et al., J. Clin. Microbiol. 43(1):414-20 (2005) or Cook et al., J. Clin. Microbiol. 42(9):4130-6 (2004)).

Ideally, though not necessarily, an infected host who is administered a composition of the invention will, eventually, exhibit no detectable HCV RNA is his body for a period of time (e.g., 12 or more weeks).

The term “no detectable HCV-RNA” in the context of the present invention means that there is less than about 100 copies of HCV-RNA per ml of serum of the patient as measured by quantitative, multi-cycle reverse transcriptase PCR (rtPCT) methodology. Such PCR based assays are conventional and very well known in the art. In general, rtPCR is performed by isolating the RNA from a specimen, reverse-transcribing it to generate cDNAs, amplifying specific nucleic acid sequences by PCR, and then using a variety of methods to detect the amplified sequences (Urdea et al., Clin. Chem. 43:1507-1511 (1997)).

In one embodiment, a composition of the present invention, when administered to a host infected with a Flaviviridae virus, will exhibit a sustained virologic response. The term “sustained virologic response” as used in the context of the present invention means that there is no detectable HCV-RNA in the serum of patients treated in accordance with the present invention for at least 24 weeks after the end of the combined therapy treatment. Preferably, the period of sustained virologic response is at least one year—or longer—after the end of treatment.

Similarly, dosage in the treatment of cancerous indications discussed herein can be monitored using methods which are well known in the art and the treating clinician or physician can adjust the dosage of the IFN-Ig being administered according to the level of progress observed and according to other clinical exigencies that are observed (e.g., adverse reactions to the chosen treatment regimen). In an embodiment of the invention, the progress of the treatment of leukemia indications as discussed herein (e.g., hairy cell leukemia or chronic myelogenous leukemia), using an IFN-Ig of the present invention, are monitored using blood chemistry tests such as for the leukocyte enzyme alkaline phosphatase (LAP score) (Rambaldi et al., Blood 73(5):1113-5 (1989)). A lower LAP score has been associate with CML. In an embodiment of the invention, the progress of the treatment of hairy cell leukemia, with an IFN-Ig of the present invention, is monitored, for example, with a complete blood count to detect a low white blood cell count, a low red cell count or low platelets; a physical examination to detect an enlarged spleen or liver; a bone marrow biopsy to detect hairy cells; a peripheral blood smear to detect hairy cells; a test done on blood or bone marrow cells for tartrate-resistant acid phosphatase which can confirm the presence of hairy cells; or an abdominal computed tomography (CT) scan to detect an enlarged spleen and liver. In an embodiment of the invention, the progress of treatment of a melanoma, using an IFN-Ig of the present invention, is monitored by visual inspection of the skin including whole body photography and mole mapping. In an embodiment of the invention, the progress of treatment of malignant osteopertosis can be monitored using skeletal X-rays (X-rays of osteopetrosis patients will often have an unusual density with a chalky white appearance), bone density tests, bone biopsies, CAT scans or MRI. In an embodiment of the invention, the progress of treatment of refractory or recurrent condylomata acuminate (genital warts) can be monitored by visual inspection of the infected skin surface. In an embodiment of the invention, the progress of treatment of follicular lymphome can be monitored by complete blood count (CBC), examination of peripheral blood smears, chest X-rays and CT scans and blood chemistries tests (e.g., including LDH, uric acid, liver function tests, and creatinine). LDH is often an indicator of tumor load wherein elevated LDH is a negative prognostic factor. In an embodiment of the invention, the progress of treatment of Kaposi's sarcoma (e.g., AIDS related Kaposi's sarcoma) is monitored by visual inspection of skin lesions, chest X-rays (to visualize lesions in the lungs), Bronchoscopy (to visualize lesions in the upper airway) and endoscopy (to visualize lesions in the stomach and small intestine). In an embodiment of the invention, the progress of treatment of infections associated with chronic granulomatous disease (CGD) is monitored by a patient interview and monitoring of bodily temperature, a chest X-ray, a blood count to detect any excessively high level of immunoglobulin, or a test to detect an elevated erythrocyte sedimentation rate or ESR (a sign of chronic infection or inflammation). In an embodiment of the invention, the progress of treatment of multiple sclerosis (MS) is monitored by magnetic resonance imaging to detect the presence of plaques or scarring in brain as well as patient interviews to monitor the frequency of attacks, exacerbations, flares, or relapses of MS symptoms.

### Kits and Articles of Manufacture

Kits of the present invention include IFN-IgG4 fusion or a pharmaceutical composition thereof, for example, in a pharmaceutical dosage form such as a pill, a powder, an injectable liquid, a tablet, dispersible granules, a capsule, a cachet or a suppository. See for example, Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

The kits of the present invention also include information, for example in the form of a package insert, including information concerning the pharmaceutical compositions and dosage forms in the kit. Generally, such information aids patients and physicians in using the enclosed pharmaceutical compositions and dosage forms effectively and safely. For example, the following information regarding IFN-IgG4 may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

The kits of the invention may also include another therapeutic composition such as ribavirin, for example, combined with a pharmaceutically acceptable carrier, in a pharmaceutical formulation, more preferably in a pharmaceutical dosage form such as a pill, a powder, an injectable liquid, a tablet, dispersible granules, a capsule, a cachet or a suppository (e.g., Rebetol®).

IFN-IgG4 and the other therapeutic composition (e.g., ribavirin) can be supplied, in the kit, as separate compositions or combined into a single composition.

### EXAMPLES

The following examples are provided to more clearly describe the present invention and should not be construed to limit the scope of the invention.

### Example 1

### Cloning, Expression and Purification of Human Interferon-Alfa-2b/Ala-Ser/Human IgG4 Fc Fusion Protein

Cloned human interferon-alfa-2b (IFNa2b), including the IFNa2b signal peptide sequence, derived from vector pE3-327-IFNa2b was fused to human IgG4 Fc via PCR reaction. The mature protein comprised human IFNa2b-(Ala-Ser linker)-human IgG4 Fc:

Human Interferon-Alfa-2b/Ala-Ser/Human IgG4 Fc Fusion Protein (SEQ ID NO: 2):

```
human IFN-alfa-2b signal sequence
  M A L T F A L L V A L L V L S
ATG GCC TTG ACC TTT GCT TTA CTA GTG GCC CTC CTG GTG CTC AGC
  C K S S C S V G
TGC AAG AGC TCC TGC AGC GTG GGC
human IFN-alfa-2b
  C D L P Q T H S L G S R R T L
TGT GAT CTG CCT CAA ACC CAC AGC CTG GGT AGC AGG AGG ACC TTG
  M L L A Q M R R I S L F S C L
ATG CTC CTG GCA CAG ATG AGG AGA ATC TCT CTT TTC TCC TGC TTG
  K D R H D F G F P Q E E F G N
AAG GAC AGA CAT GAC TTT GGA TTT CCC CAG GAG GAG TTT GGC AAC
  Q F Q K A E T I P V L H E M I
CAG TTC CAA AAG GCT GAA ACC ATC CCT GTC CTC CAT GAG ATG ATC
  Q Q I F N L F S T K D S S A A
CAG CAG ATC TTC AAT CTC TTC AGC ACA AAG GAC TCA TCT GCT GCT
  W D E T L L D K F Y T E L Y Q
TGG GAT GAG ACC CTC CTA GAC AAA TTC TAC ACT GAA CTC TAC CAG
  Q L N D L E A C V I Q G V G V
CAG CTG AAT GAC CTG GAA GCC TGT GTG ATA CAG GGG GTG GGG GTG
  T E T P L M K E D S I L A V R
ACA GAG ACT CCC CTG ATG AAG GAG GAC TCC ATT CTG GCT GTG AGG
  K Y F Q R I T L Y L K E K K Y
AAA TAC TTC CAA AGA ATC ACT CTC TAT CTG AAA GAG AAG AAA TAC
  S P C A W E V V R A E I M R S
AGC CCT TGT GCC TGG GAG GTT GTC AGA GCA GAA ATC ATG AGA TCT
  F S L S T N L Q E S L R S K E
TTT TCT TTG TCA ACA AAC TTG CAA GAA AGT TTA AGA AGT AAG GAA
linker
  A S
GCT AGC
human IgG4 Fc
  D K T H T C P P C P A P E F L
GAC AAA ACT CAC ACA TGC CCA CCA TGC CCA GCA CCT GAG TTC CTG
  G G P S V F L F P P K P K D T
GGG GGA CCA TCA GTC TTC CTG TTC CCC CCA AAA CCC AAG GAC ACT
  L M I S R T P E V T C V V V D
CTC ATG ATC TCC CGG ACC CCT GAG GTC ACG TGC GTG GTG GTG GAC
  V S Q E D P E V Q F N W Y V D
GTG AGC CAG GAA GAC CCC GAG GTC CAG TTC AAC TGG TAC GTG GAT
  G V E V H N A K T K P R E E Q
GGC GTG GAG GTG CAT AAT GCC AAG ACA AAG CCG CGG GAG GAG CAG
  F N S T Y R V V S V L T V L H
TTC AAC AGC ACG TAC CGT GTG GTC AGC GTC CTC ACC GTC CTG CAC
  Q D W L N G K E Y K C K V S N
CAG GAC TGG CTG AAC GGC AAG GAG TAC AAG TGC AAG GTC TCC AAC
  K G L P S S I E K T I S K A K
AAA GGG CTC CCG TCC TCC ATC GAG AAA ACC ATC TCC AAA GCC AAA
  G Q P R E P Q V Y T L P P S Q
GGG CAG CCC CGA GAG CCA CAG GTG TAC ACC CTG CCC CCA TCC CAG
  E E M T K N Q V S L T C L V K
GAG GAG ATG ACC AAG AAC CAG GTC AGC CTG ACC TGC CTG GTC AAA
  G F Y P S D I A V E W E S N G
GGC TTC TAC CCC AGC GAC ATC GCC GTG GAG TGG GAG AGC AAT GGG
  Q P E N N Y K T T P P V L D S
CAG CCG GAG AAC AAC TAC AAG ACC ACG CCT CCC GTG CTG GAC TCC
  D G S F F L Y S R L T V D K S
GAC GGC TCC TTC TTC CTC TAC AGC AGG CTA ACC GTG GAC AAG AGC
  R W Q E G N V F S C S V M H E
AGG TGG CAG GAG GGG AAT GTC TTC TCA TGC TCC GTG ATG CAT GAG
  A L H N H Y T Q K S L S L S L
GCT CTG CAC AAC CAC TAC ACA CAG AAG AGC CTC TCC CTG TCT CTG
  G K
GGT AAA TGA
linkers for other forms
  A S G S G (SEQ ID NO: 7)
GCT AGC GGA TCC GGC (SEQ ID NO: 21)
  A S G S G S G (SEQ ID NO: 8)
GCT AGC GGC AGC GGA TCC GGC (SEQ ID NO: 22)
  A S G G G G S G G G G S G G G G S G (SEQ ID NO: 17)
GCT AGC GGAGGCGGTGGATCCGGTGGAGGCGGCAGTGGTGGTGGAGGAAGCGGC (SEQ ID NO: 23)
```

The bold, underscored residue in the IgG4 region is a mutated residue which is serine in wild-type IgG4. The mutation facilitates the formation of intermolecular disulfide bonds between the IgG4 molecules (thus favoring creation of dimeric forms of the fusions) and hampers formation of intramolecular bonding.

The vector included an ampicillin-resistant marker and cytomegalovirus promoter region.

DNA was prepared by transforming plasmids into Escherichia coli XL1-Blue (Stratagene), growth for 10 hours in 500 mL Luria Broth-50 ug/mL ampicillin, and dsDNA preparation using Qiagen Plasmid Maxi Kit (Qiagen, Catalog # 12163). Purified dsDNA was transfected into human embryonic kidney 293 (HEK293) cells using the calcium phosphate method (Gorman et al., DNA Prot. Engineer. Tech. 2:3 (1990)). Cells were initially grown in 50% Hamm's F12/50% DMEM F-12 with 10% fetal bovine serum (Cellgro). Twenty-four hours after transfection the media was changed to CHO-PF serum-free (Sigma) with 1 ug/mL apo-transferrin (Sigma) and 5 ug/mL insulin (Sigma) added. The secreted protein was harvested 96 hours after transfection. Supernatent was applied to a protein A-Sepharose CL-4B column (Pharmacia), buffer exchanged with phosphate-buffered saline (PBS) and concentrated to 0.5 mL using a Centriprep-10 (Amicon). Purified protein was quantitated by A₂₈₀.

Additional fusion proteins were generated by inserting linkers of various sizes between the Ala-Ser dipeptide sequence and the human IgG4 Fc. These linkers were: Gly-Ser-Gly (SEQ ID NO: 9), Gly-Ser-Gly-Ser-Gly (SEQ ID NO: 10), and (Gly-Gly-Gly-Ser)₃-Gly (SEQ ID NO: 11).

### Example 2

### Pharmacokinetics of IFN Alfa-2b-IgG4

Human IFN alfa-2b-IgG4 fusion constructs were prepared using conventional molecular cloning techniques. Briefly, three constructs were prepared: one comprising a direct fusion between the C-terminus of IFN alfa-2b, and Ala-Ser linker, and the Fc region of human IgG4 (CH2+CH3+hinge region) with the remaining two containing linkers comprising ASGSG (SEQ ID NO: 7) or ASGSGSG (SEQ ID NO: 8). Following transfection of HEK293 cells, the expressed recombinant proteins were purified over Protein-A and solubilized in an PBS (phosphate buffer saline) buffer at concentrations ranging between 0.79 and 2.31 mg/mL. The bioactivity (IFN α2b-related) of these fusion proteins was assessed using a validated bioassay. Reference native IFN alfa-2b standard protein was used to compare the bioactivity of the fusion proteins. The pharmacokinetics of these fusion proteins was evaluated in Sprague Dawley rats following intravenous administration at a dose of 1 mg/kg. Plasma samples obtained pre-dose, 1, 4, 8, 24 hours, and day 2, 3, 4, 7, 10, 14, 22, and 28 post-dose were analyzed using a validated bioassay. The results of the assays are set forth below in Table 2.

TABLE 2
Bioactivity of each fusion over time.

| | SCH X | SCH Y | SCH Z |
|---|---|---|---|
| | (AS linker) | (ASGSG linker) | (ASGSGSG linker) |
| Time, days | Bioactivity (IU/mL) | | |
| 0 (pre-dose) | NS^{a} | NS^{a} | NS^{a} |
| 0.042 | 9600 | 9600 | 19200 |
| 0.17 | 4800 | 9600 | 9600 |
| 0.33 | 2400 | 9600 | 9600 |
| 1 | 1200 | 4800 | 4800 |
| 2 | 2400 | 4800 | 2400 |
| 3 | 2400 | 2400 | 2400 |
| 4 | 2400 | 2400 | 1200 |
| 7 | 1200 | 2400 | 1200 |
| 10 | 600 | 2400 | 1200 |
| 14 | 1200 | 1200 | 1200 |
| 22 | 600 | 600 | 300 |
| 28 | NS^{a} | NS^{a} | NS^{a} |

^{a}NS = no sample available due to sample clotting

The bioactivity of the 3 fusion constructs ranged between 2.1 and 3.3×10⁶ IU/mg protein (IFN reference standard bioactivity is 2.6×10⁸ IU/mg protein; as determined in an in vitro assay).

The cytopathic effect (CPE) assay with which the interferon bioactivity was measured was peformed is based on the ability of interferon (IFN) (or molecules with IFN activity) to protect cells from virus-induced cell death or cytopathic effect. The assay used human fibroblast cells (FS-71, normal human diploid foreskin cells) and EMC virus (encephalomyocarditis virus) in a 96-well format: a fixed number of cells were cultured in the presence of samples (and a reference IFN standard) containing IFN for about 4 hours followed by infection with a fixed number of virus particles. After virus-infected control cells reach a pre-determined stage of CPE, the media was harvested from all cells and assayed colorimetrically for intracellular markers indicative of intact cells using crystal violet. The samples were then compared to the IFN reference standard activity curve for calculation of bioactivity.

The pharmacokinetics of these fusion proteins in rats demonstrated a substantial improvement in the terminal half-life (t½) of IFN-derived bioactivity relative to the IFN protein. The t½ ranged between 5.6 to 9.7 days compared to a t½ of 1 hour with the IFN protein in rats (t½ is ˜2 hours in humans).

### Example 3

### Expression, Purification and Characterization of IFNa2b-IgG4

The production cell line, 293 c18, was obtained from the American Type Culture Collection (CRL-10852) and was maitained in DMEM supplemented with 10% FBS. The cDNAs encoding the IFN fusion proteins, SCH Y and SCH Z, were cloned into expression vector pCEP4 vector (Invitrogen Corp.; Carlsbad, Calif.). The expression vectors, pCEP4-LPD475 and pCEP4-LPD476, were transfected into 293 c18 cells using TransIT-293 (Mirus Bio, Madison, Wis.). Transfected cell culture was then treated with Geneticin (400 μg/mL) and Hygromycin B (400 μg/mL).

For the production of the fusion proteins, the transfected cells were seeded into T-flasks in DMEM with 10% FBS. Approximately three days post seeding, the culture was replaced with 293 SFMII (Invitrogen Corp.) supplemented with 200 mM glutamine (40 ml/L), Trace A (1 ml/L), Trace B (1 ml/L), Tris Ph 7.4 (15 ml/L), IS (iron supplement) Fe (1 ml/L, Irvine Scientific; Santa Ana, Calif.). Within 72 hours, the temperature of the incubator was reduced from 37° C. to 34° C. and was maintained at 34° C. throughout the production. The culture supernatant was recovered at approximately 10-12 days after the temperature switch. The conditioned culture media was centrifuged with a tabletop centrifuge. The supernatant was filtered through a 0.2 μm filter.

The fusions were purified by Protein A column chromatography as follows: two clones of IFNa2b-IgG4 were purified using a 5 mL HiTrap-rProtein A column. The column dimensions were 1.6 cm×2.5 cm. The purifications were carried out in the cold room using AKTA 100 system (GE Healthcare; Piscataway, N.J.). The loading target was approximately 10 mg/mL of resin.

The column was equilibrated with three column volumes of 10 mM sodium phosphate with 125 mM sodium chloride, pH 7.2. Then the feed was loaded on the column at a flow rate of 1 ml/min. The column was washed with ten column volumes of the above buffer at the same flow rate. After the wash, the bound protein was eluted using 100 mM sodium acetate, pH 2.9 at a flow rate of 1 ml/min.

The pool was pH adjusted to 5.5 using 1M Tris base, filtered through 0.22 μm filter and used for preliminary analysis. Later the pool was dialyzed against a liter of 10 mM sodium phosphate with 125 mM sodium chloride, pH 7.2, 0.22 μm, filtered and stored at 4° C.

TABLE 3
Analytical results of the pools:

| | | Protein | Total | % yield | | Total | | % dimer | % presumed | Endotoxin |
|---|---|---|---|---|---|---|---|---|---|---|
| | Vol | by RP-HPLC | Protein (mg) | by | Protein | Protein (mg) | % yield | from | multimer from | level |
| Sample | (mL) | (mg/mL) | by RP-HPLC | RP_HPLC | by A280 | by A280 | by A280 | SE-HPLC | SE-HPLC | (EU/mg) |
| IFN2αFc | 19.6 | 2.19 | 42.9 | 75 | 2.6 | 51.0 | 89.4 | 96% | 4% | 1.9 |
| SCH Y | | | | | | | | | | |
| IFN2αFc | 19.6 | 1.71 | 33.5 | 69 | 1.8 | 35.3 | 72.4 | 97% | 3% | 2 |
| SCH Z | | | | | | | | | | |

The IFNa2b-IgG4 fusion was analyzed on a size exclusion HPLC column and the results are set forth below in Table 4.

TABLE 4
Size Exclusion HPLC analysis of IFNa2b-IgG4.
Sequence MW IFNa2b-IgG4 (SCH Z) = 45070.15
Sequence MW IFNa2b-IgG4 (SCH Y) = 45214.28

| Calibration | | Log | Time of | Calibration | | Log | Time of |
|---|---|---|---|---|---|---|---|
| of SEC-HPLC | MW | MW | elution | of SEC-HPLC | MW | MW | elution |
| lysozyme | 14300 | 4.16 | 10.800 | lysozyme | 14300 | 4.16 | 10.800 |
| BSA | 66200 | 4.82 | 8.875 | BSA | 66200 | 4.82 | 8.875 |
| IgG | 147736 | 5.17 | 8.000 | IgG | 147736 | 5.17 | 8.000 |
| IFNa2b-IgG4 | 95600 | 4.98 | 8.465 | IFNa2b-IgG4 | 96631 | 4.99 | 8.453 |
| SCH-Y | | | | SCH-Z | | | |

Lysozyme, bovine serum albumin (BSA) and IgG were run on the column as size standards against which the size of IFNa2b-IgG4 SCH-Y or SCH-Z was calculated. Since the molecular weight is approximately half of the size of the polypeptide eluted from the column, it was deduced that the IFNa2b-IgG4 eluted from the column was a dimer.

The bioactivity of the fusions expressed in this example were measured as set forth above in an in vitro cytopathic effect (CPE) assay:
IFNa2b-IgG4 (SCH Y): 7.48×10⁶ IU/mg
IFNa2b-IgG4 (SCH Z): 1.02×10⁷ IU/mg

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. An isolated polypeptide comprising one or more interferon polypeptides fused to one or more IgG4 Fc polypeptides.

2. The polypeptide of claim 1 wherein the interferon is a member selected from the group consisting of is interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon gamma, interferon alpha F and interferon alpha con-1.

3. The polypeptide of claim 1 comprising the amino acid sequence set forth in a member selected from the group consisting of SEQ ID NOs: 2, 3 and 15.

4. The polypeptide of claim 1 wherein the interferon comprises an amino acid sequence selected from the group consisting of SEQ ID NOs: 12-14.

5. The polypeptide of claim 1 wherein the IgG4 comprises an amino acid sequence set forth in SEQ ID NO: 1.

6. The polypeptide of claim 1 wherein the interferon is fused to the IgG4 by a peptide linker.

7. The polypeptide of claim 6 wherein the linker comprises from about 2 to about 18 amino acids.

8. The polypeptide of claim 6 wherein the linker comprises an amino acid sequence selected from the group consisting of:

| | |
|---|---|
| Ala Ser Gly Ser Gly; | (SEQ ID NO: 7) |
| Ala Ser Gly Ser Gly Ser Gly; | (SEQ ID NO: 8) |

9. A multimer comprising two or more polypeptides of claim 1 optionally coordinated with a divalent cation.

10. The multimer of claim 9 wherein the cation is Zn²⁺.

11. The polypeptide of claim 1 which is crystalline.

12. A pharmaceutical composition comprising the polypeptide of claim 1 and a pharmaceutically acceptable carrier.

13. A composition comprising the polypeptide of claim 1 in association with one or more further pharmaceutical agents or a pharmaceutical composition thereof.

14. A composition comprising the polypeptide of claim 1 in association with one or more further pharmaceutical agents suitable for treating a medical condition selected from the group consisting of flaviviridae virus infection, multiple sclerosis, serious infections associated with chronic granulomatous disease, malignant osteopetrosis, refractory or recurring external condylomata acuminate, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, hepatitis B infection and hepatitis C infection or a pharmaceutical composition thereof.

15. The composition of claim 14 wherein the additional pharmaceutical agent is a member selected from the group consisting of ribavirin, isatoribine, VX-497, viramidine, BILN 2061, VX-950 and IDN-6556.

16. An isolated polynucleotide encoding a polypeptide of claim 1.

17. The polynucleotide of claim 16 comprising a nucleotide sequence selected from the group consisting of SEQ ID NOs: 4, 5 and 16.

18. An isolated vector comprising the polynucleotide of claim 16.

19. An isolated host cell comprising the vector of claim 18.

20. A method for increasing the in vivo half-life of interferon comprising fusing the interferon to IgG4.

21. The method of claim 20 wherein the interferon fused to the IgG4 comprises an amino acid sequence selected from the group consisting of SEQ ID NOs: 2, 3 and 15.

22. The method of claim 20 wherein the interferon is a member selected from the group consisting of interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon gamma, interferon alpha F and interferon alpha con 1.

23. The method of claim 22 wherein the interferon comprises an amino acid sequence selected from the group consisting of SEQ ID NOs: 12-14.

24. The method of claim 20 wherein the IgG4 comprises an amino acid sequence set forth in SEQ ID NO: 1.

25. A method for treating or preventing, in a subject, a medical condition selected from the group consisting of flaviviridae virus infection, multiple sclerosis, serious infections associated with chronic granulomatous disease, malignant osteopetrosis, refractory or recurring external condylomata acuminate, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, hepatitis B infection, hepatitis C infection, and any medical condition treatable by interferon therapy comprising administering, to the subject, a therapeutically effective amount of an isolated polypeptide comprising interferon fused to IgG4 or a pharmaceutical composition thereof.

26. The method of claim 25 wherein the interferon fused to IgG4 comprises an amino acid sequence selected from the group consisting of SEQ ID NOs: 2, 3 and 15.

27. The method of claim 25 wherein the subject is pregnant or a nursing mother.

28. The method of claim 25 wherein the interferon is a member selected from the group consisting of interferon alfa-1a, interferon alfa-1b, interferon alfa-2a, interferon alfa-2b, interferon alfa-2c, interferon alfa-4a, interferon alfa-4b, interferon alfa-5, interferon alfa-6, interferon alfa-7a, interferon alfa-7b, interferon alfa-8a, interferon alfa-8b, interferon alfa-8c, interferon alfa-10a, interferon alfa-10b, interferon alfa-13, interferon alfa-14a, interferon alfa-14b, interferon alfa-14c, interferon alfa-16, interferon alfa-17a, interferon alfa-17b, interferon alfa-17c, interferon alfa-17d, interferon alfa-21a, interferon alfa-21b, interferon alfa-24, interferon beta, interferon omega, interferon tau, interferon alfa-N3, interferon beta-1a, interferon beta-1b, interferon gamma-1b, interferon gamma, interferon alpha F and interferon alpha con1.

29. The method of claim 25 wherein the polypeptide is administered in association with one or more further pharmaceutical agents or a pharmaceutical composition thereof.

30. The method of claim 29 wherein the polypeptide is administered in association with one or more further pharmaceutical agents suitable for treating a medical condition selected from the group consisting of flaviviridae virus infection, multiple sclerosis, serious infections associated with chronic granulomatous disease, malignant osteopetrosis, refractory or recurring external condylomata acuminate, hairy cell leukemia, chronic phase, Philadelphia chromosome (Ph) positive chronic myelogenous leukemia (CML), malignant melanoma, follicular lymphoma, condylomata acuminata, AIDS-related kaposi's sarcoma, hepatitis B infection and hepatitis C infection or a pharmaceutical composition thereof.

31. The method of claim 30 wherein the further pharmaceutical agent is selected from the group consisting of ribavirin, isatoribine, VX-497, viramidine, BILN 2061, VX-950 and IDN-6556.

32. The method of claim 25 wherein the interferon comprises an amino acid sequence selected from the group consisting of SEQ ID NOs: 12-14.

33. The method of claim 25 wherein the IgG4 comprises an amino acid sequence set forth in SEQ ID NO: 1.

34. The method of claim 25 wherein the host is a human.

35. The method of claim 25 wherein the medical condition is hepatitis C infection and wherein therapeutically effective amount of the isolated polypeptide comprising interferon fused to IgG4, optionally in association with an anti-viral therapeutic, or the pharmaceutically acceptable composition thereof, is administered for a treatment time period sufficient to eradicate detectable hepatitic C virus-RNA and to maintain no detectable hepatitic C virus RNA for at least twelve weeks after the end of the treatment time period.

36. A method for making a polypeptide comprising interferon fused to IgG4 comprising introducing a polynucleotide of claim 16 into a host cell under conditions wherein the polynucleotide is expressed.

37. The method of claim 36 further comprising isolating the polypeptide.

38. A polypeptide produced by the method of claim 36.

39. The method of claim 36 wherein the interferon fused to IgG4 comprises an amino acid sequence selected from the group consisting of SEQ ID NOs: 2, 3 and 15.


SEQUENCE LISTING (25 sequences)

SEQ ID NO: 1 - protein, 227 aa, Artificial Sequence
  IgG4
DKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 417 aa, Artificial Sequence
  interferon-alfa-2b-human IgG4 Fc fusion protein
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEASDKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 395 aa, Artificial Sequence
  human interferon-alfa-2a-human IgG4 Fc fusion protein
MCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEASDKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - DNA, 1251 bp, Artificial Sequence
  human interferon-alfa-2b-human IgG4 Fc
atggcnytnacnttygcnytnytngtngcnytnytngtnytnwsntgyaarwsnwsntgywsngtnggntgygayytnccncaracncaywsnytnggnwsnmgnmgnacnytnatgytnytngcncaratgmgnmgnathwsnytnttywsntgyytnaargaymgncaygayttyggnttyccncargargarttyggnaaycarttycaraargcngaracnathccngtnytncaygaratgathcarcarathttyaayytnttywsnacnaargaywsnwsngcngcntgggaygaracnytnytngayaarttytayacngarytntaycarcarytnaaygayytngargcntgygtnathcarggngtnggngtnacngaracnccnytnatgaargargaywsnathytngcngtnmgnaartayttycarmgnathacnytntayytnaargaraaraartaywsnccntgygcntgggargtngtnmgngcngarathatgmgnwsnttywsnytnwsnacnaayytncargarwsnytnmgnwsnaargargcnwsngayaaracncayacntgyccnccntgyccngcnccngarttyytnggnggnccnwsngtnttyytnttyccnccnaarccnaargayacnytnatgathwsnmgnacnccngargtnacntgygtngtngtngaygtnwsncargargayccngargtncarttyaaytggtaygtngayggngtngargtncayaaygcnaaracnaarccnmgngargarcarttyaaywsnacntaymgngtngtnwsngtnytnacngtnytncaycargaytggytnaayggnaargartayaartgyaargtnwsnaayaarggnytnccnwsnwsnathgaraaracnathwsnaargcnaarggncarccnmgngarccncargtntayacnytnccnccnwsncargargaratgacnaaraaycargtnwsnytnacntgyytngtnaarggnttytayccnwsngayathgcngtngartgggarwsnaayggncarccngaraayaaytayaaracnacnccnccngtnytngaywsngayggnwsnttyttyytntaywsnmgnytnacngtngayaarwsnmgntggcargarggnaaygtnttywsntgywsngtnatgcaygargcnytncayaaycaytayacncaraarwsnytnwsnytnwsnytnggnaar

SEQ ID NO: 5 - DNA, 1182 bp, Artificial Sequence
  human interferon-alfa-2a-human IgG4 Fc
tgygayytnccncaracncaywsnytnggnwsnmgnmgnacnytnatgytnytngcncaratgmgnaarathwsnytnttywsntgyytnaargaymgncaygayttyggnttyccncargargarttyggnaaycarttycaraargcngaracnathccngtnytncaygaratgathcarcarathttyaayytnttywsnacnaargaywsnwsngcngcntgggaygaracnytnytngayaarttytayacngarytntaycarcarytnaaygayytngargcntgygtnathcarggngtnggngtnacngaracnccnytnatgaargargaywsnathytngcngtnmgnaartayttycarmgnathacnytntayytnaargaraaraartaywsnccntgygcntgggargtngtnmgngcngarathatgmgnwsnttywsnytnwsnacnaayytncargarwsnytnmgnwsnaargargcnwsngayaaracncayacntgyccnccntgyccngcnccngarttyytnggnggnccnwsngtnttyytnttyccnccnaarccnaargayacnytnatgathwsnmgnacnccngargtnacntgygtngtngtngaygtnwsncargargayccngargtncarttyaaytggtaygtngayggngtngargtncayaaygcnaaracnaarccnmgngargarcarttyaaywsnacntaymgngtngtnwsngtnytnacngtnytncaycargaytggytnaayggnaargartayaartgyaargtnwsnaayaarggnytnccnwsnwsnathgaraaracnathwsnaargcnaarggncarccnmgngarccncargtntayacnytnccnccnwsncargargaratgacnaaraaycargtnwsnytnacntgyytngtnaarggnttytayccnwsngayathgcngtngartgggarwsnaayggncarccngaraayaaytayaaracnacnccnccngtnytngaywsngayggnwsnttyttyytntaywsnmgnytnacngtngayaarwsnmgntggcargarggnaaygtnttywsntgywsngtnatgcaygargcnytncayaaycaytayacncaraarwsnytnwsnytnwsnytnggnaar

SEQ ID NO: 6 - protein, 23 aa, Artificial Sequence
  interferon signal sequence
MALTFALLVALLVLSCKSSCSVG

SEQ ID NO: 7 - protein, 5 aa, Artificial Sequence
  linker peptide
ASGSG

SEQ ID NO: 8 - protein, 7 aa, Artificial Sequence
  linker peptide
ASGSGSG

SEQ ID NO: 9 - protein, 3 aa, Artificial Sequence
  linker peptide
GSG

SEQ ID NO: 10 - protein, 5 aa, Artificial Sequence
  linker peptide
GSGSG

SEQ ID NO: 11 - protein, 13 aa, Artificial Sequence
  linker peptide
GGGSGGGSGGGSG

SEQ ID NO: 12 - protein, 166 aa, Artificial Sequence [sequence omitted]

SEQ ID NO: 13 - protein, 166 aa, Artificial Sequence
  human interferon alfa-2a
MCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 14 - protein, 167 aa, Artificial Sequence
  human consensus interferon alpha (alpha con-1)
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 15 - protein, 396 aa, Artificial Sequence
  human interferon-alpha con-1-human IgG4 Fc fusion protein
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKEASDKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 1188 bp, Artificial Sequence
  human interferon-alpha con-1-human IgG4 Fc
atgtgygayytnccncaracncaywsnytnggnaaymgnmgngcnytnathytnytngcncaratgmgnmgnathwsnccnttywsntgyytnaargaymgncaygayttyggnttyccncargargarttygayggnaaycarttycaraargcncargcnathwsngtnytncaygaratgathcarcaracnttyaayytnttywsnacnaargaywsnwsngcngcntgggaygarwsnytnytngaraarttytayacngarytntaycarcarytnaaygayytngargcntgygtnathcargargtnggngtngargaracnccnytnatgaaygtngaywsnathytngcngtnaaraartayttycarmgnathacnytntayytnacngaraaraartaywsnccntgygcntgggargtngtnmgngcngarathatgmgnwsnttywsnytnwsnacnaayytncargarmgnytnmgnmgnaargargcnwsngayaaracncayacntgyccnccntgyccngcnccngarttyytnggnggnccnwsngtnttyytnttyccnccnaarccnaargayacnytnatgathwsnmgnacnccngargtnacntgygtngtngtngaygtnwsncargargayccngargtncarttyaaytggtaygtngayggngtngargtncayaaygcnaaracnaarccnmgngargarcarttyaaywsnacntaymgngtngtnwsngtnytnacngtnytncaycargaytggytnaayggnaargartayaartgyaargtnwsnaayaarggnytnccnwsnwsnathgaraaracnathwsnaargcnaarggncarccnmgngarccncargtntayacnytnccnccnwsncargargaratgacnaaraaycargtnwsnytnacntgyytngtnaarggnttytayccnwsngayathgcngtngartgggarwsnaayggncarccngaraayaaytayaaracnacnccnccngtnytngaywsngayggnwsnttyttyytntaywsnmgnytnacngtngayaarwsnmgntggcargarggnaaygtnttywsntgywsngtnatgcaygargcnytncayaaycaytayacncaraarwsnytnwsnytnwsnytnggnaar

SEQ ID NO: 17 - protein, 18 aa, Artificial Sequence
  linker peptide
ASGGGGSGGGGSGGGGSG

SEQ ID NO: 18 - protein, 13 aa, Artificial Sequence
  linker peptide
GSGSGSGSGSGSG

SEQ ID NO: 19 - protein, 15 aa, Artificial Sequence
  linker peptide
ASGGGSGGGSGGGSG

SEQ ID NO: 20 - protein, 2 aa, Artificial Sequence
  linker peptide
AS

SEQ ID NO: 21 - DNA, 15 bp, Artificial Sequence
  DNA encoding peptide linker
gctagcggatccggc

SEQ ID NO: 22 - DNA, 21 bp, Artificial Sequence
  DNA encoding peptide linker
gctagcggcagcggatccggc

SEQ ID NO: 23 - DNA, 54 bp, Artificial Sequence
  DNA encoding peptide linker
gctagcggaggcggtggatccggtggaggcggcagtggtggtggaggaagcggc

SEQ ID NO: 24 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 25 - protein, 407 aa, Artificial Sequence
  human IL-10-IgG4 fusion
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNASDKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK